(19) United States
(12) Patent Application Publication
(10) Pub. No.: US 2026/0305455 A1
(43) Pub. Date: Oct. 1, 2026

(54) MICROELECTRONICS DEVICE PACKAGE INTEGRATING MULTIPLE DIES

(71) Applicant: Texas Instruments Incorporated, Dallas, TX (US)

(72) Inventors: Enis Tuncer, Dallas, TX (US); Rongwei Zhang, Plano, TX (US)

(21) Appl. No.: 19/096,331

(22) Filed: Mar. 31, 2025

(51) Int. Cl. H10W 74/10; G01N 33/00; H10W 74/01; H10W 70/40; H10W 80/00; H10W 90/00

(52) U.S. Cl. CPC H10W 74/124; G01N 33/0027; H10W 74/01; H10W 70/417; H10W 80/312; H10W 80/327; H10W 90/792

(57) ABSTRACT

An apparatus includes: a first die having devices formed within or over a device side surface of a substrate and further comprising contacts extending from a backside surface; a second die mounted over a die pad of a package substrate, the package substrate further comprising leads spaced from the die pad, the second die comprising a semiconductor die having bond pads on a device side surface configured for mounting the first die, and having additional bond pads; the first die mounted to the second die by die-to-die bonds formed between the contacts and the bond pads; electrical connections between additional bond pads on the device side surface of the second die and leads of the package substrate; and mold compound covering at least a portion of the first die, the second die, the electrical connections, a portion of the die pad and a portion of the leads.

### TECHNICAL FIELD

This disclosure relates generally to microelectronic device packages, and more particularly to microelectronic device packages multiple semiconductor dies or components integrated together in a single microelectronic device package.

### BACKGROUND

Processes for producing microelectronic device packages include mounting one or more semiconductor dies to a package substrate and subsequently covering the electronic devices with a dielectric material, such as a mold compound, to form packaged microelectronic devices.

Incorporating multiple dies in a microelectronic device package is increasing. These microelectronic device packages can be referred to as “multichip modules” or “MCMs,” or as “system-in-package” or “SIP” devices. Example applications include packaging multiple dies together in a system using dies that are coupled together to perform a desired function. In an example application, a sensor die can be combined with another semiconductor die, which can be a controller or processor associated with the sensor die. In one example sensor type, metal oxide gas sensor dies can be packaged together with associated semiconductor dies to increase integration, increase performance and reduce the system board area required. Other examples can include a first die and a second die, where the combined dies form a system. Storage devices such as volatile memory or non-volatile memory can be combined with a microprocessor or controller semiconductor die. Passive components such as resistor, inductors or capacitors can be combined with semiconductor dies including active circuitry to form a system in a package. Integrating the semiconductor dies together into a microelectronic device package can also include integrating the passive components needed for a commonly needed function to act as a single component on a system board. Increasing integration by packaging passive components and the associated semiconductor dies together in a single microelectronic device package can increase ease of use for an end user and can reduce system board design time.

Prior approaches to packaging a first semiconductor die with a second semiconductor die include forming a stacked die package. Electrical connections between the first semiconductor die and the second semiconductor die may be made with die-to-die wire bonding from the first semiconductor die, an upper die when the package is placed in a normal orientation, to a second semiconductor die, a lower semiconductor die in a normal orientation, and then using additional wire bonds to connect additional bond pads of the second semiconductor die to leads of a package substrate.

In an alternative prior approach, the semiconductor dies are packaged together using a multi-chip module (“MCM”) package substrate such as a laminate substrate or printed circuit board with conductor traces is used. The first semiconductor die and the second semiconductor die are mounted near or adjacent to one another on the package substrate. The first semiconductor die and the second semiconductor die can be coupled to one another by conductive traces or by wire bonds, the system is then packaged, with the side-by-side layout of the two semiconductor dies resulting in a larger overall microelectronic device package (when compared with a stacked die configuration such as described above.) Both prior approaches require multiple assembly steps at the die level, advanced wire bonding steps or package substrates with conductive traces and are relatively expensive package solutions.

A continuing need thus exists for a robust and economical microelectronic device package with multiple semiconductor dies integrated together.

### SUMMARY

In a described example, a method includes: forming a first die having a device side surface, and having contacts extending from a backside surface opposite the device side surface, at least some of the contacts electrically coupled to conductors within or over the first die; mounting the first die on a device side surface of a second die, the contacts of the first die forming a bond with bond pads on the device side surface of the second die; mounting the first die and the second die on a device mounting surface of a package substrate, the package substrate further comprising leads spaced from the device mounting surface; forming electrical connections between additional bond pads on the second die and the leads of the package substrate; and covering a portion of the first die, the second die, the electrical connections, and a portion of the leads with mold compound, the mold compound forming a body of a microelectronic device package.

In an additional described example, a method includes: forming a sensor die having a layer of sensor material on a top surface, and having contacts extending from a backside surface opposite the top surface, at least some of the contacts electrically coupled to electrodes configured to sense electrical changes in the sensor layer; mounting the sensor die on a device side surface of a semiconductor die, the contacts of the sensor die forming a bond with bond pads on the device side surface of the semiconductor die; mounting the sensor die and semiconductor die on a device mounting surface of package substrate, the package substrate further comprising leads spaced from the device mounting surface; forming electrical connections between bond pads on the semiconductor dies and the leads of the package substrate; and covering a portion of the sensor die, the semiconductor die, and the electrical connections with mold compound, and covering a portion of the leads with the mold compound, the mold compound forming a body of a microelectronic device package with an open cavity extending into the body exposing the layer of sensor material.

In a further described example, a method includes: forming a gas sensor die on a substrate, the gas sensor die comprising a metal oxide sensor material over electrodes on a device side surface of the substrate, and further comprising contacts extending from a backside surface of the substrate, the electrodes electrically coupled to corresponding ones of the contacts; forming a semiconductor die on a semiconductor substrate, the semiconductor die having bond pads on a device side surface configured for mounting the gas sensor die; mounting the semiconductor die on a die pad of a package substrate, the package substrate further comprising leads spaced from the die pad; mounting the gas sensor die on the device side surface of the semiconductor die, the contacts forming a die-to-die bond with the bond pads on the device side surface of the semiconductor die; forming wire bond connections between additional bond pads on the semiconductor die and the leads of the package substrate; and covering a portion of the gas sensor die, the semiconductor die, and the electrical connections with mold compound, and covering a portion of the die pad and the leads with the mold compound, the mold compound forming a body of a microelectronic device package with an open cavity extending into the body exposing the metal oxide sensor material.

In an additional described example, an apparatus includes: a first die having devices formed within or over a device side surface of a substrate and further comprising contacts extending from a backside surface of the substrate opposite the device side surface; a second die mounted over a die pad of a leadframe, the leadframe further comprising leads spaced from the die pad, the second die comprising a semiconductor die having bond pads on a device side surface configured for mounting the first die, and having additional bond pads; the first die mounted to the second die by die-to-die bonds formed between the contacts and the bond pads configured for mounting the first die; wire bond connections between additional bond pads on the device side surface of the second die and leads of the package substrate; and mold compound covering at least a portion of the first die, the second die, the wire bond connections, a portion of the die pad and a portion of the leads, the mold compound forming a body of a microelectronic device package.

In another described example, an apparatus includes: a semiconductor die mounted to a device mounting surface of a package substrate, the semiconductor die having bond pads on a device side surface; a sensor die having a layer of sensor material on a top surface, and having contacts extending from a backside surface opposite the top surface, the sensor die mounted using a die-to-die bond between the contacts of the sensor die and the bond pads of the semiconductor die; electrical connections formed between additional bond pads on the device side surface of the semiconductor die and the leads of the package substrate; and mold compound covering a portion of the sensor die, the semiconductor die, the electrical connections, and portions of the leads, and forming a body of a microelectronic device package with an open cavity extending into the body exposing the layer of sensor material.

In a further described example, a microelectronic device package for a gas sensor includes: mounting a semiconductor die on a die pad of a leadframe, the leadframe further comprising leads spaced from the die pad, the semiconductor die having bond pads on a device side surface configured for mounting a sensor die; a gas sensor die comprising a metal oxide sensor material and electrodes over a top side surface of a substrate and further comprising contacts extending from a backside surface of the substrate, the electrodes electrically coupled to corresponding ones of the contacts, the gas sensor die mounted to the device side surface of the semiconductor die by forming a die-to-die bond between the contacts and the bond pads on the semiconductor die; wire bond connections between additional bond pads on the device side surface of the semiconductor die and leads of the package substrate; and mold compound covering a portion of the gas sensor die, the semiconductor die, and the wire bond connections, the mold compound covering a portion of the die pad and the leads, the mold compound forming a body of a microelectronic device package with an open cavity extending into the body exposing a portion of the metal oxide sensor material.

### BRIEF DESCRIPTION OF THE DRAWINGS

FIGS. 1A-1B illustrate, in cross-sectional views, a microelectronic device package including a first example arrangement, and an alternative microelectronic device package including a second example arrangement, respectively.

FIGS. 2A-2B illustrate, in additional cross-sectional views, an alternative microelectronic device package including an example flip-chip arrangement, and an alternative microelectronic device package including an example of another flip-chip arrangement, respectively.

FIGS. 3A-3B illustrate, in a projection view and a close-up projection view, respectively, semiconductor dies on a semiconductor wafer and an individual semiconductor die from the semiconductor wafer and arranged for wire bonding. FIG. 3C illustrates an alternative individual semiconductor die arranged for flip-chip mounting.

FIGS. 4A-4M illustrate, in a series of cross-sectional views, selected steps for forming a sensor die of an example arrangement.

FIGS. 5A-5C illustrate, in another series of cross-sectional views, selected steps for mounting a sensor die of the arrangements to a semiconductor die. FIGS. 5D-5G illustrate, in a further set of cross-sectional views, steps for packaging the sensor die and the semiconductor die of an example arrangement in a microelectronic device package.

FIG. 6 illustrates, in a flow diagram, selected steps of a method for forming an arrangement.

FIG. 7 illustrates, in an additional flow diagram, selected steps of a method for forming a sensor die of the arrangements.

FIG. 8 illustrates, in a further flow diagram, selected steps of a method for forming a microelectronic device package incorporating a sensor die and a semiconductor die of the arrangements.

### DETAILED DESCRIPTION

Corresponding numerals and symbols in the different figures generally refer to corresponding parts, unless otherwise indicated. The figures are not necessarily drawn to scale.

Elements are described herein as “coupled.” The term “coupled” includes elements that are directly connected and elements that are indirectly connected, and elements that are electrically connected even with intervening elements or wires are coupled.

The term “semiconductor die” is used herein. A semiconductor die can be a discrete semiconductor device such as a bipolar transistor, a few discrete devices such as a pair of power FET switches fabricated together on a single semiconductor die, or a semiconductor device can be an integrated circuit with multiple semiconductor devices such as the multiple capacitors in an A/D converter. The semiconductor die can include passive devices such as resistors, inductors, filters, sensors, or active devices such as transistors. The semiconductor die can be an integrated circuit with hundreds or thousands of transistors coupled to form a functional circuit, for example a microprocessor or memory device. When semiconductor devices are fabricated contemporaneously on a semiconductor wafer and then individually separated from the semiconductor wafer and from one another, the individual units are referred to as “semiconductor dies.” A semiconductor device die is also a semiconductor device. In an example arrangement, a sensor die and a corresponding semiconductor die, which may implement a controller for the sensor die, are manufactured independently in wafer fabrication facilities, and then are combined in a vertically stacked combination which can be incorporated into a single microelectronic device package. In alternative arrangements, a first die and a second die are vertically arranged in a stacked combination and can be incorporated into a single microelectronic device package.

The term “passive component” is used herein. As used herein, a passive component is a component without active devices, for example, a resistor, capacitor, inductor, coil, diode, or certain sensors.

The term “die” is used herein. A “die” is a unit device fabricated on a substrate in a group or gang manufacturing operation. In an example process, the substrate can be a semiconductor substrate, such as a silicon wafer. The dies produced are then semiconductor dies. However, in some example arrangements, certain dies for sensors can be formed on other substrates, such as glass or ceramic.

The term “sensor die” is used herein. A sensor die can be a semiconductor die with a sensor material formed on a surface. The sensor die can be formed, in some examples, on a semiconductor substrate using semiconductor fabrication processes. However, in some alternative examples, a sensor die can be formed on a carrier substrate that is not a semiconductor, such as glass or ceramic material. In an example arrangement that is described below, a metal oxide gas sensor is formed as an example sensor die. The metal oxide gas sensor includes a sensor material layer of metal oxide material with electrodes on a surface. For the metal oxide sensor material, heater conductors can also be fabricated in dielectric material positioned beneath the metal oxide layer. In operation, when the layer of metal oxide is heated to an elevated temperature and then exposed to an oxidizing gas which increases, or decreases, the oxygen at the surface of the metal oxide layer, the resistance of the metal oxide layer increases. The resistance change can be detected using electrodes, and the change can be used to detect the gas. Certain gases are oxidizing and increase the oxygen, and in those example applications, the resistance increases with gas concentration. In another application, when the metal oxide layer is heated to an operation temperature and then exposed to a volatile organic compound (“VOC”), the oxygen is reduced at the surface of the metal oxide layer, the resistance again changes, now decreasing, and using the electrodes, the change can be detected, indicating the presence of a VOC gas. An example of VOC gas that can be detected is ethanol. Metal oxide layers for use in metal oxide sensors can include SnO₂, ZnO, TiO₂, CoO, and other metal oxides. Gases that can be detected include nitrogen, hydrogen, water vapor, carbon monoxide, carbon dioxide and various VOC gases.

A sensor die can also be a die having other types of sensors that use a sensor material layer to sense changes in conditions. Examples include acoustic wave sensors, which can use aluminum nitride (AlN) layers, for example, to sense certain conditions. Acoustic wave material is a piezoelectric material that changes electrically with pressure, stress, or strain, for example. Conductors in interdigitated patterns can be formed either within (for bulk acoustic wave or “BAW” sensors) or on (for surface acoustic wave or “SAW” sensors) the piezoelectric layer. For example, an aluminum nitride layer can be used, which is a polycrystalline layer. Other examples of sensors useful in the arrangements include photosensors, temperature, relative humidity, and acoustic sensors. Liquid sensors can be used to detect carbonation levels or concentrations in liquids. Water/rainfall sensors can be formed using conductors on a dielectric layer that are spaced apart, the water providing a conductive path between electrodes on the sensor layer, which can close an electronic circuit and thereby provide a signal.

In certain applications using sensor dies, in operation, the sensor material is exposed to the ambient atmosphere. A microelectronic device package for the sensor can have an open cavity that exposes at least a portion of the sensor material layer, so that the atmosphere, light, sound, liquid, or gas being sensed is in contact with the sensor material layer. In an example using a metal oxide layer as a gas sensor, the microelectronic device package exposes the metal oxide layer to the atmosphere in a cavity opening in the package body. In the arrangements, the package body can be formed using a mold compound. In one example approach for a sensor application, during molding a cavity opening is formed on one surface of the molded package that is aligned with the sensor material layer on the surface of the sensor die. In operation, the cavity opening in the package allows the atmosphere to contact the sensor material layer on the sensor die. In another example application for a relative humidity sensor, a polymer dielectric that has a dielectric constant or capacitance that changes with changes in humidity can be used, for example a polyimide layer can be used as the sensor material layer.

In example arrangements, a first die is mounted on a second die. In this description, the first die, which can be an upper die (the upper die when the elements are oriented with the device side surface of the second die facing upwards), can have backside contacts extending from a backside surface. The second die can have bond pads on a device side surface that are positioned to correspond to the backside contacts of the first die, and the first die can be mounted to the second die by forming die-to-die bonds between the backside contacts and the bond pads on the second die. In the arrangements, the combined first die and second die can be mounted to a package substrate, and electrical connections can be formed between additional bond pads on the second die and leads of the package substrate. In example arrangements, the second die can be a semiconductor die. The first die can be another semiconductor die, a sensor die, a passive component, or another device that has backside contacts. Mold compound is used to form a molded body for a microelectronic device package including the combined first and second dies.

The term “microelectronic device package” is used herein. As used herein, a microelectronic device package has at least one semiconductor die electrically coupled to terminals and has a package body that protects and covers the at least one semiconductor die. The microelectronic device package can include additional semiconductor dies or additional elements. In certain example arrangements, a first die, which can be for example a passive component, a sensor die, or a semiconductor die, and a second die, for example another semiconductor die, are vertically positioned and couped together, and are packaged together. In additional example arrangements a semiconductor die, and a sensor die that is mounted to and coupled to the semiconductor die are packaged together. In example arrangements, multiple semiconductor dies can be packaged together without a sensor die, or alternatively multiple sensor dies can be packaged together. In one approach, a first semiconductor die (which can be, in some examples, a sensor die) is mounted to a second semiconductor die. The second semiconductor die or dies can be mounted to die pads of a package substrate, and electrical connections are formed between bond pads on the second semiconductor die and leads of the package substrate. In some examples, wire bonds can be used to form the electrical connections. In an alternative approach, after the first semiconductor die is positioned and mounted to the second semiconductor die, the second semiconductor die can then be flip chip mounted to conductive lands on a package substate. Solder balls or conductive post connects extending from the device side surface of the second die can then be used to form the electrical connections that couple the second die to the package substrate.

The term “package substrate” is used herein. A package substrate is a substrate arranged to receive a semiconductor die and in the illustrated example arrangements, other components, and to support the semiconductor die in a completed semiconductor device package. Package substrates useful with the arrangements include conductive leadframes, molded interconnect substrates (MIS), partially etched leadframes, pre-molded leadframes (PMLFs), embedded trace substrates (ETS), and multilayer package substrates. In certain example arrangements, the package substrate can be a leadframe. In additional example arrangements, the package substrate can be a flip chip package substrate. Certain leads of the package substrate are configured as terminals of the microelectronic device package by exposing portions of the leads from the mold compound.

In packaging semiconductor dies and related components such as sensor dies together, mold compound may be used to partially cover a package substrate, to entirely cover the package substrate, to cover passive components, to cover semiconductor dies, to cover sensor dies, and to cover the electrical connections made to the package substrate. This molding process is often referred to as an “encapsulation” process, although portions of the package substrates are often not covered in the mold compound during encapsulation; for example, terminals for the microelectronic device package can be formed by leaving portions of conductive leads exposed from the mold compound. The terminals are configured for making electrical connections to the microelectronic device package. Encapsulation is often a transfer molding process, where a thermoset mold compound, such as an epoxy resin, can be used. A room temperature solid or powdered epoxy resin mold compound can be positioned within a mold tool and heated to a liquid state, and then molding can be performed by pressing the now liquid mold compound into a mold through runners or channels. Transfer molding can be used. Unit molds shaped to surround an individual device may be used, or a block molding process may be used. The molding process is typically used to form multiple packages simultaneously for several devices from mold compound. The devices to be molded can be provided in an array or matrix of several, hundreds or even thousands of devices in rows and columns on a package substrate strip or grid, the devices can then be molded contemporaneously, reducing per unit costs and increasing throughput.

After the molding process is complete, the individual microelectronic device packages are cut apart from each other in a sawing operation. A mechanical saw can be used to cut through the mold compound and any remaining package substrate material in saw streets that are formed between the molded devices. Portions of the package substrate leads are exposed from the mold compound package to form terminals for the microelectronic device packages.

For example arrangements that use leaded packages, a trim and form tool can be used to separate temporary supports, or “tie bars” from the leads, and to form the leads into the desired shape, such as a gull-wing shape, for surface mounting to a board.

The term “scribe lane” is used herein. A scribe lane is a portion of semiconductor wafer between semiconductor dies. Sometimes in related literature the term “scribe street” is used. Once semiconductor processing is finished and the semiconductor devices are complete, the semiconductor devices are separated into individual semiconductor dies by severing the semiconductor wafer along the scribe lanes. The separated dies can then be removed and handled individually for further processing. This process of removing dies from a wafer is referred to as “singulation” or sometimes referred to as “dicing.” Scribe lanes are arranged on four sides of semiconductor dies and when the dies are singulated from one another, rectangular semiconductor dies are formed.

The term “saw street” is used herein. A saw street is an area between molded electronic devices used to allow a saw, such as a mechanical blade, laser, or other cutting tool to pass between the molded electronic devices to separate the devices from one another. This process is another form of singulation. When the molded electronic devices are provided in a strip with one device adjacent to another device along the strip, the saw streets are parallel and normal to the length of the strip. When the molded electronic devices are provided in an array of devices in rows and columns, the saw streets include two groups of parallel saw streets, the two groups are normal to each other, and the saw will traverse the molded electronic devices in two different directions to cut apart the packaged electronic devices from one another in the array.

In example arrangements, a first die has metal contacts extending from a backside surface. The metal contacts can be electrically coupled to devices formed within the first die. The first die can be mounted on a device side surface of a second die using bond pads on the second die, with the metal contacts and the bond pads forming die-to-die electrical and mechanical bonds. In one example method for forming arrangements, the first die is mounted to the second die after the second die is mounted to a package substate. In an alternative example method for forming additional arrangements, a die-to-wafer bonding process mounts the first dies to a second semiconductor dies which, at the bonding point in the assembly process, are still part of a semiconductor wafer, the combined elements can then be singulated from the semiconductor wafer and then packaged as units.

In certain applications, sensor dies are used in an arrangement as the first die. In a particular example, metal oxide layers on the sensor die can be used to sense the presence of certain gases. A metal oxide layer has electrical properties that can change in a predictable way under certain conditions, the electrical change can be measured to form a sensor. In an example application for a gas sensor, when in operation, the metal oxide is heated to an elevated operational temperature between 200-400 degrees Celsius, and a control circuit in a second die can be used to register a baseline measurement. The baseline measurement can be stored and used to compare to new measurements. The second die can include circuitry or processors configured to determine and quantify a measurement in a data form corresponding to units or quantities a system or user can make use of. The gas sensor in the first die includes the metal oxide layer over a heater formed of one or more resistive heater conductors, and power is applied to the heater to warm the metal oxide layer to an operating temperature.

Sometimes a metal oxide layer which changes resistivity with changes in oxygen concentration at the surface of the layer is referred to as a “chemistor.” Changes in the conductivity or resistivity of the metal oxide layer can be observed by providing sense electrodes spaced apart by or positioned spaced apart within the metal oxide layer. When an oxygen reducing gas is present, the oxygen concentration at the surface is reduced, which will change the resistivity of the heated metal oxide layer, the changed resistivity can be compared to a baseline value and the presence of a gas can be detected. Example gases are a volatile organic compound (“VOC”) gas (which reduces oxygen at the surface by consuming it), the reducing action decreases the resistance of the metal oxide (“MOX”) layer, and in contrast, oxidizing gases such as nitrogen which increase oxygen at the surface can be detected. When an oxidizing gas such as nitrous oxide is present, the oxygen concentration at the surface of the MOX layer will increase due to “donor” oxygen atoms from the oxidizing gas, and again a change in the resistivity can be detected. As the oxygen concentration increases, the resistance of the metal oxide layer also increases.

Metal oxides that can be used in gas sensors include, for example, tin oxide (SnO₂), titanium oxide (TiO), tungsten oxide (WO₂), indium oxide (In₂O₃), zinc oxide (ZnO), and cobalt oxide (CoO). In an example integrated device, the controller semiconductor die can include non-volatile or volatile data storage such as EEPROM, FLASH or SRAM memory, where calibration data and baseline measurements can be stored, in addition the controller semiconductor device can provide power FET devices for supplying current to the heater strips and power to the sensor. A/D converters, comparators, amplifiers, and filters can be included in the semiconductor die to improve measurements and to process or format the data before outputting a sensed value in a form that is useful to the system, such as digital data corresponding to a measurement.

In an example arrangement, a first semiconductor die can be a sensor die formed on a semiconductor or other substrate, for example a silicon wafer can be used. In an example process useful in forming sensors of an arrangement, a backside etch process can be performed on the substrate to expose metal contacts extending from a backside surface of the sensor dies. These metal contacts can form terminals for the sensor die. The metal contacts can be coupled with heater conductors and/or to sensor electrodes of the sensor dies, forming the electrical terminals for the sensor dies. In metal oxide gas sensor examples, the sensor dies have a layer of metal oxide on a device side surface, opposite the backside surface with the exposed metal contacts. When other sensor material layers are used, the heater conductors may be unnecessary and can be omitted for these applications.

After the sensor dies are removed from the wafer or substrate, the first die, in this example an individual sensor die, is then mounted on a device side surface of a second die, which can be a semiconductor die. The backside metal contacts of the first die directly contact bond pads on the semiconductor die to form a die-to-wafer or a die-to-die bond. In an example mounting process, a bond is formed between the metal contacts of the first die, and conductive pads on the second die, which can be of the same or compatible materials. Examples include aluminum, copper, gold, and silver. Copper to aluminum bonds can be formed, or vice versa, as well as same metal bonds. When different metals are bonded, intermetallic compounds can be formed as part of the bonds, whereas when same metals are bonded, a copper-copper, gold-gold, silver-silver, or aluminum-aluminum bond can form. Solder can be applied to the bond pads and a solder joint can be formed. Silver sintering can be used to bond the devices.

Example arrangements include sensor dies with a sensor material layer, such as a crystal layer, a piezoelectric layer, a polymer dielectric, or a metal oxide layer, formed so that when the sensor material layer is exposed to a condition or to a substance to be detected, an electrical change occurs in the sensor material layer. The electrical change can be measured using electrodes coupled to or positioned within the sensor material layer, and thus the condition or presence of the substance can be detected, or the amount of the substance can be measured. Metal oxide layers can be used, for example, to detect gases such as oxidizing gases including nitrogen, and to detect reducing gases such as volatile organic compounds, for example ethanol, in an environment. Acoustic wave sensors can use crystal or polycrystalline layers to detect changes in frequency vibrations that correlate to temperature, pressure, strain, adsorption or desorption, viscosity of certain liquids, dew point or humidity. The acoustic wave sensor materials can be piezoelectric materials. Aluminum nitride can be used for acoustic wave sensor materials. Humidity sensors can be formed using a polymer dielectric that has a dielectric constant or capacitance that changes with humidity. Temperature sensors can include a sensor material that changes resistivity with temperature. Photodetectors can include a photosensitive sensor layer. Acoustic wave sensors can include bulk acoustic wave (“BAW”) sensors and surface acoustic wave (“SAW”) sensors. Electrodes in acoustic wave sensor material, for example aluminum nitride (AlN), can generate acoustic waves in or on the surface of the sensor material. As the sensor material is exposed to different conditions or substances, the frequency, phase, or amplitude of the acoustic waves change in predictable ways. These changes can be measured and converted into electrical signals that indicate changes in temperature, pressure, strain, or the presence of certain gases.

Methods useful for forming the arrangements include wafer scale processes for forming sensor dies in parallel, reducing costs. A substrate, which can be a silicon wafer, another semiconductor wafer, a silicon dioxide (glass) or a ceramic substrate, is provided. On a device side surface of the substrate, an array of sensor dies are formed. In a process useful with the arrangements, trenches (later used for forming the backside metal contacts) are formed extending into the device side surface of the carrier substrate. Metal is deposited in the trenches, the trenches having a pointed bottom surface. An insulator layer is deposited over the metal in the trenches. In a particular process for producing metal gas sensors as the sensor dies, heater conductors are deposited on the insulator layer and can be patterned in strips. Another layer of insulator or dielectric material is deposited over the heater conductors. Sensing electrode traces can be deposited over the insulator layer and patterned to form electrodes spaced from one another. A metal oxide layer can be deposited over the electrode traces.

The substrate is then processed in a back end of the line (“BEOL”) or assembly/test (“A/T”) facility in a backside process. In one example process useful for forming the arrangements, a backside etch is performed that is selective to the metal contacts, to partially remove the substrate material from the bottom surface of the substrate while exposing the pointed bottom portions of the metal contacts. The substrate is then singulated along scribe streets between unit devices on the substrate in a wafer dicing process to separate the metal oxide gas sensor dies one from another. Each metal oxide sensor die has a metal oxide layer on a top or device side surface, and the metal contacts extending from the opposite backside surface.

In an example assembly method using die-to-wafer bonding, a semiconductor wafer is processed to form the second semiconductor dies. In particular examples the second semiconductor dies can be controller circuits corresponding to sensor dies. In an example process for assembling first semiconductor dies that include heater electrodes such as metal oxide gas sensor dies to second semiconductor dies, the semiconductor wafer for the second semiconductor dies has thermal isolation trenches formed on the device side surface of each individual second semiconductor die in locations corresponding to mounting positions for the metal oxide gas sensor dies. Bond pads or conductive lands are formed on either side of the thermal isolation trenches, the bond pads are configured for mounting the backside metal contacts of the metal oxide gas sensor dies.

Automated pick-and-place equipment can then be used to mount the metal oxide gas sensor dies to the semiconductor wafer. After the metal gas sensor dies are placed, a bond is formed between the bond pads of the semiconductor dies and the metal contacts of the metal gas sensor dies. In some example processes, the metal contacts bond directly to the bond pads, using either the same metal material for both the metal contacts and the bond pads, or using metals that form a useful intermetallic compound bond, such as aluminum metal contacts bonded to copper bond pads. In another example process, solder can be used to form a solder joint, and a thermal reflow process may be used. The first dies, which can be sensor dies in certain examples, are now mounted to the second dies which, in some example processes, can be semiconductor dies that remain in wafer form for part of the assembly process. The wafer can then be singulated by dicing along scribe lanes between the individual second die units to provide individual combined units ready for subsequent packaging processes.

While in one approach to forming the arrangements the first dies, such as sensor dies, are mounted to the second dies while still in wafer form (as described above), in an alternative approach for forming example arrangements, the first dies can be mounted to previously singulated second dies, which can be semiconductor dies, after a die mounting process mounts the second dies to a package substrate using a die attach material. In this approach the first dies can be mounted to the second dies using die-to-die bonding, instead of die-to-wafer bonding as described above.

The combined first and second dies can be mounted to a package substrate, such as a multilayer package substrate, laminate, or a leadframe. (Alternatively, the second dies, which can be semiconductor dies, can be mounted to a leadframe first, in a die attach process. The first dies can then be mounted to the second dies using the metal contacts on the backside of the sensor dies.) Electrical connections, for example wire bonds, are formed between additional bond pads of the second dies and the leads of the package substrate. Mold compound can be used to form a package body over the second dies, the electrical connections, portions of the package substrate, and the first dies. In an example arrangement for a sensor, the first die is a sensor die, and the package body formed by the mold compound has an open cavity that allows at least a portion of the sensor die to be exposed to the atmosphere. In further examples, the first die can be another semiconductor die, and no open cavity is needed or formed for these applications.

Use of the arrangements provides an economical, wafer scale process for producing first dies with backside metal contacts and uses existing assembly equipment such as pick-and-place tools, die-to-die bonding, and molding tools to form an economical microelectronic device package for the combined first die and second die.

An advantage of the backside metal contacts of the first dies in the arrangements is that using a small, pointed backside contact on the first dies allows for a die-to-die bond to be formed without the need for compressive mechanical pressure on a sensor layer, which can be a thin layer of sense material that is possibly subject to damage if a higher mechanical pressure were applied to the sensor die in the assembly process.

In additional arrangements, the first die and the second die can be semiconductor dies that have various functions. For example, a first die can be a memory or storage device, while the second die can have a circuit that is coupled and configured to access the storage, such as a microcontroller, microprocessor, programmable circuit, or filter. In an additional example the first die can be a passive component, such as a resistor, inductor, capacitor, or diode, and the second die can be coupled with the first die to form a circuit. Additional examples include arrangements where a first semiconductor die and a second semiconductor die are identical or have similar functions, such as in arrangements forming memory modules, where to extend the size of a memory module in a microelectronic device package, multiple semiconductor dies can be stacked vertically using the backside metal contacts of the arrangements.

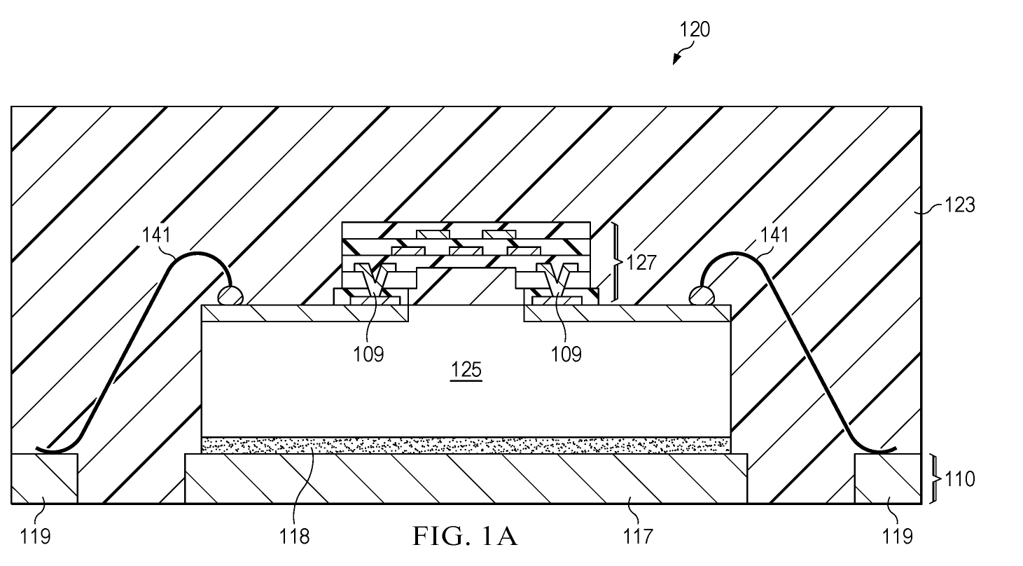

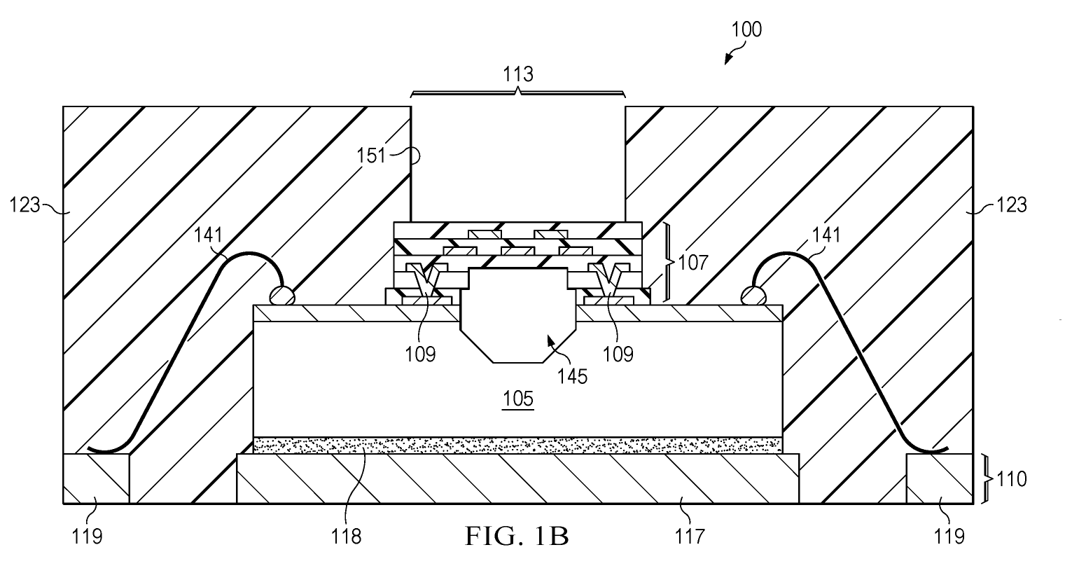

FIG. 1A illustrates, in a cross-sectional view, an example arrangement of a microelectronic device package 120. A first die 127 is shown, which can be a semiconductor die. A second die 125, which can also be a semiconductor die, is shown with the first die 127 mounted to the device side surface of the second semiconductor die 125. The second semiconductor die 125 is mounted on a die pad 117 of package substrate 110, which can be a leadframe, for example. Die attach material 118 is used to mount the second semiconductor die 125 to the leadframe. Wire bonds 141 connect bond pads of the second semiconductor die 125 to the package substrate 110. Mold compound 123 covers the first die 127, the second die 125, the wire bonds 141 which form electrical connections, and portions of the package substrate 110. The package substrate 110 has leads 119 with portions exposed from the mold compound 123 that form terminals of the microelectronic device package 120. Metal contacts 109, extending from the first die 127 on a backside surface, form die-to-die bonds between the first die 127 and the second die 125.

FIG. 1B illustrates, in a cross-sectional view, another example arrangement in a microelectronic device package 100 using a sensor die as the first semiconductor die, and a semiconductor die as the second die. A sensor die 107, for example a metal oxide gas sensor die, is bonded to a semiconductor die 105. The second die, semiconductor die 105, is mounted to a package substrate 110, which in the illustrated example is a leadframe with die pad 117 configured to support the second semiconductor die 105. Die attach material 118 is used to mount the second die 105 to the die pad 117, a die attach film or die attach epoxy can be used. Conductive die attach material can be used in examples where the semiconductor die is to be electrically connected to the die pad 117. In other examples, non-conductive die attach materials can be used when the semiconductor die 105 is to be electrically isolated from the die pad 117. The package substrate 110 includes leads 119 that have portions that are exposed from the package body to form terminals. In the illustrated example a no-lead package, such as a dual flat no-lead (DFN) or quad flat no-lead (QFN) package is shown. Other package types, including leaded packages, can be used. Wire bonds 141 are shown forming electrical connections that couple the semiconductor die 105 to the leads 119. Mold compound 123 is shown covering the sensor die 107 in part, the second die 105, and the wire bonds 141. An open cavity 113 is shown with sides 151 extending into a top side surface (the top side surface as the elements are oriented in FIG. 1, in a normal orientation, with the top side surface facing upwards) of the mold compound 123. A trench 145 is shown in the device side surface of the semiconductor die 105, which provides thermal isolation between the heated gas sensor die 107 and the semiconductor die 105. In a gas sensor operation, the gas sensor die 107 will be heated to temperatures more than 200 degrees C, and the semiconductor die 105 needs to be thermally isolated from it. The backside metal contacts 109 are shown and make bonds with bond pads of the semiconductor die 105.

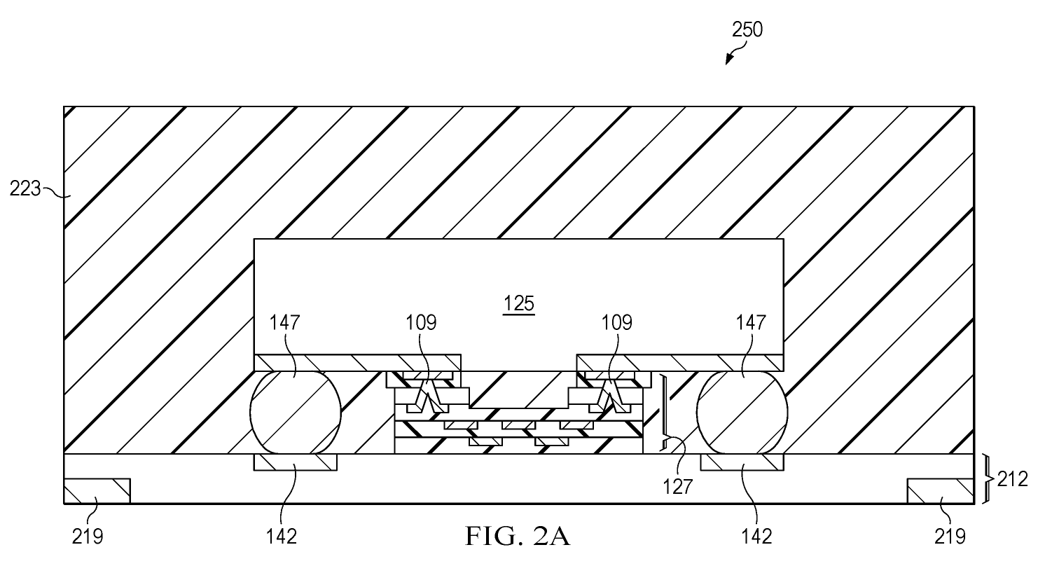

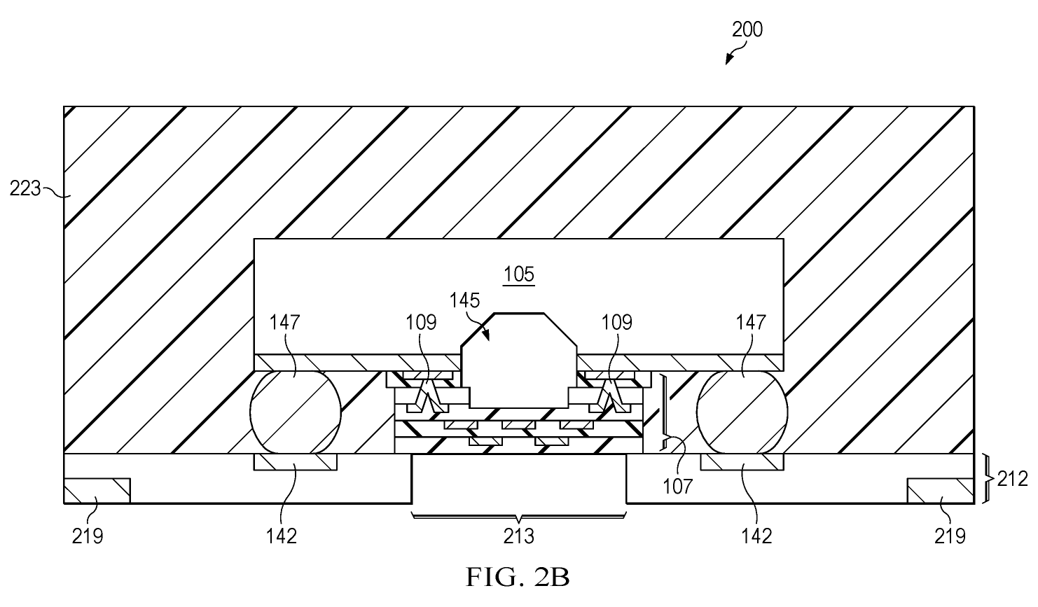

FIG. 2A illustrates, in another cross-sectional view, an alternative arrangement for a microelectronic device package 220. In FIG. 2A, the semiconductor die 125 is the same as in FIG. 1A, except that in FIG. 2A the electrical connections between the semiconductor die 125 and the package substrate 212 are made by solder balls 147 on package substrate pads 142, as a result the semiconductor die 125 has a device side surface facing a board side of the microelectronic device package 220, which is a “flip chip” package type. The microelectronic device package 220 has leads 219 of the package substrate 212. The first die 127 is the same as in FIG. 1A but is now mounted in a “possum” style on the board side of the microelectronic device package 220. Package substrate 212 can be a multilayer package substrate such as a laminate or build-up package substrate. In some arrangements the build-up package substrate can be formed using additive manufacturing by plating conductor layers and then disposing dielectric films over the conductor layers, curing the dielectric film, and then grinding the dielectric film to expose the conductor layers on a surface, this repeated process can form a multilayer build-up substrate with vertical and horizontal traces. Ajinomoto Build-up Film (“ABF”) commercially available from Ajinomoto Company Inc. of Tokyo Japan can be used to form the package substrate 212. The package substrate 212 can also be a premolded leadframe (“PMLF”) or molded interconnect substrate (“MIS”) or can be a flip chip on leads (“FCOL”) leadframe. The metal contacts 109 extend from the first die 127 and are used to form the die-to-die bonds between the first die 127 and the second die 125.

FIG. 2B illustrates, in another cross-sectional view, an alternative arrangement for a microelectronic device package 200. In FIG. 2B, the semiconductor die 105 can be the same as in FIG. 1B, except that the electrical connections between the semiconductor die 105 and the package substrate 212 are made by solder balls 147 on package substrate pads 142, the semiconductor die 105 has a device side surface facing a board side of the microelectronic device package 200, which is a “flip chip” package type, similar to the arrangement of FIG. 2A. The microelectronic device package 200 has leads 219 of the package substrate 212. The sensor die 107 is the same as in FIG. 1B and is now mounted in a “possum” style on the bottom or board side of the microelectronic device package 200, which now has the open cavity 213 facing the board side. In operation the sensor die 107 has a sensor material layer that is exposed to the atmosphere by using the open cavity 213, in the same manner as operation of the open cavity 113 in FIG. 1B.

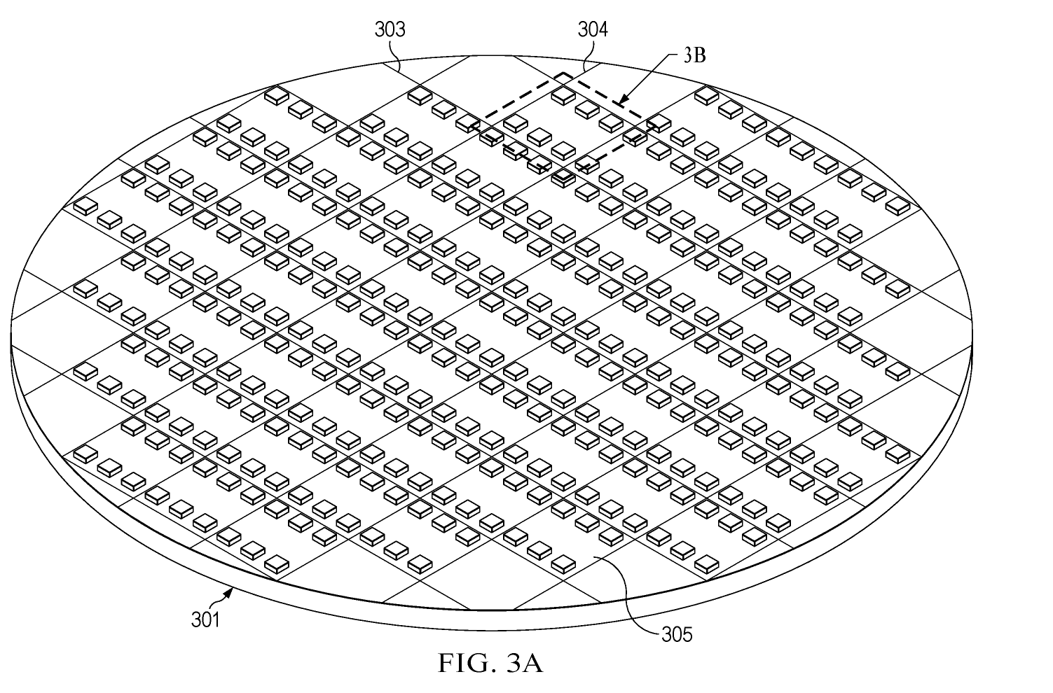

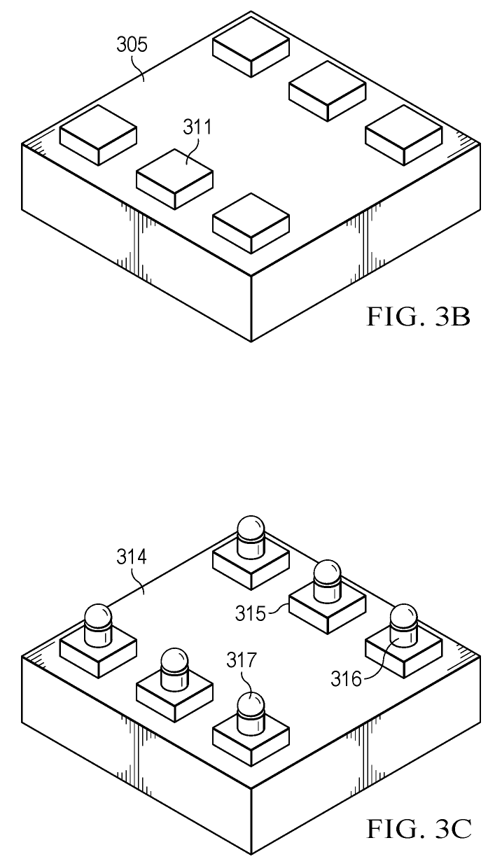

FIGS. 3A and 3B illustrate, in two projection views, a semiconductor wafer 301 having semiconductor dies 305 formed on it that are configured for wire bonding, and an individual semiconductor die 305 from the wafer 301, respectively. In FIG. 3A, semiconductor wafer 301 is shown with an array of semiconductor dies 305 formed in rows and columns on a surface. Semiconductor dies 305 can be formed using selected processes in a semiconductor wafer manufacturing facility, including ion implantation, doping, anneals, oxidation, dielectric and metal deposition, photolithography, pattern, etch, chemical mechanical polishing (CMP), electroplating, and other processes for making semiconductor devices. In FIG. 3A, scribe lanes 303 and 304, which are perpendicular to one another, and which run in parallel groups across the semiconductor wafer 301, separate the rows and columns of the completed semiconductor dies 305, and provide areas for dicing the wafer 301 to separate the semiconductor dies 305 from one another.

FIG. 3B illustrates a single semiconductor die 305 taken from semiconductor wafer 301. Semiconductor die 305 includes bond pads 311, which are conductive metal pads that are electrically coupled to devices (not shown) formed in the semiconductor die 305. Not shown for clarity of illustration are under-bump metallization (“UBM”) portions which can be formed over the bond pads 311 to improve plating and adhesion between the bond pads and ball bonds of bond wire to be formed on the bond pads 102 in processes described later herein. The semiconductor die 305 can be used in a wire bonded package, for example, by making ball bond connections on the bond pads 311.

FIG. 3C illustrates, in another projection view, a semiconductor die 314 that can be functionally similar to the semiconductor die 305 of FIGS. 3A-3B, but in FIG. 3C semiconductor die 314 is arranged for flip chip mounting. Bond pads 315 are shown with conductive post connects 316 formed on and extending from the bond pads 315, and solder bumps 317 are shown formed on the distal ends of the conductive post connects 316. In a particular example useful with the arrangements, the conductive post connects 316 can be copper pillars formed in a copper plating process at the wafer stage, and the solder bumps 317 can be formed by a solder ball drop and thermal reflow process, the resulting connections are sometimes referred to as “copper pillar bumps.” However other conductive materials and different methods can be used, gold or other conductors can be used to form the conductive post connects, and solder can be placed using other methods to form the solder bumps 317 on the distal ends of conductive post connects 316.

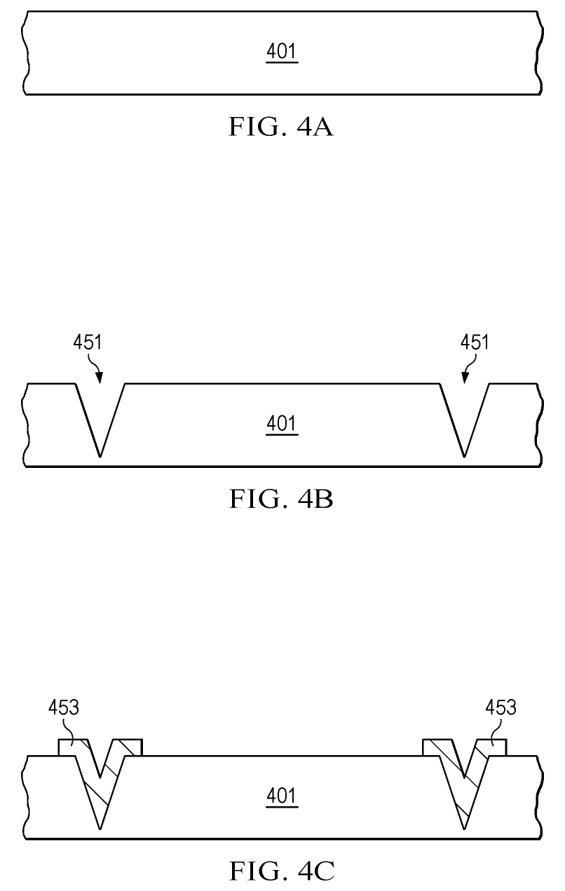

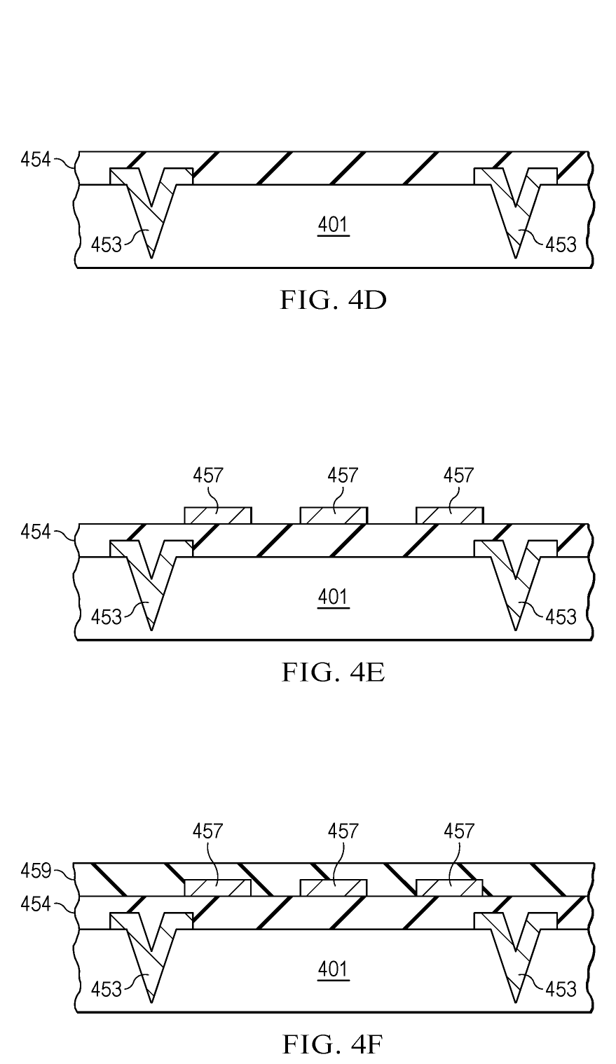

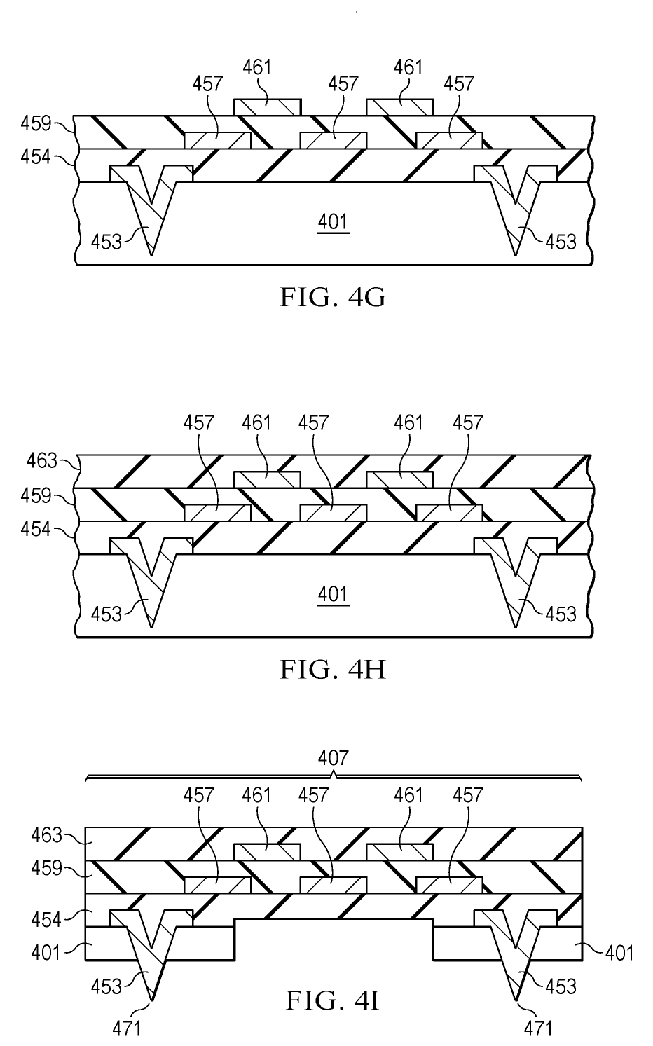

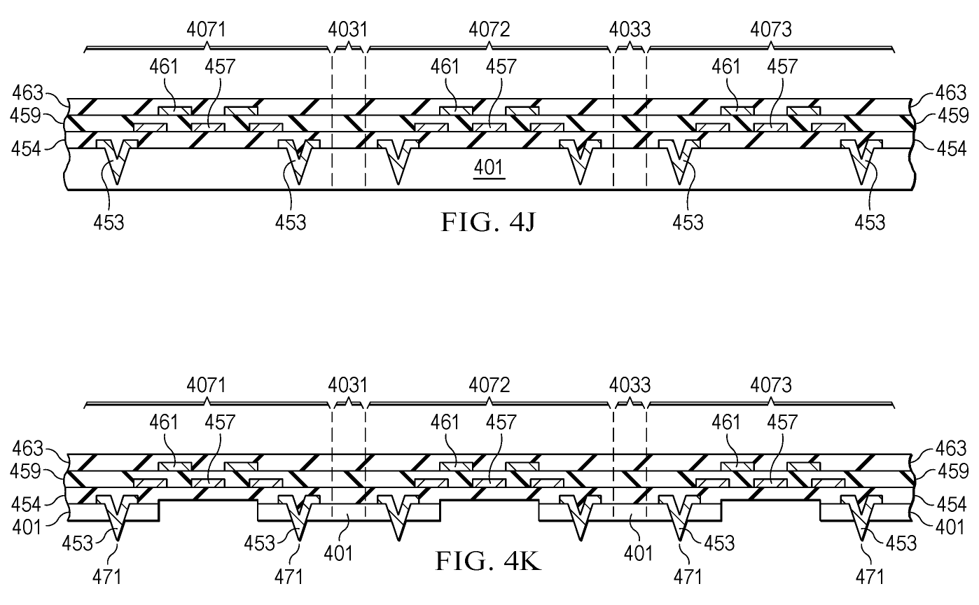

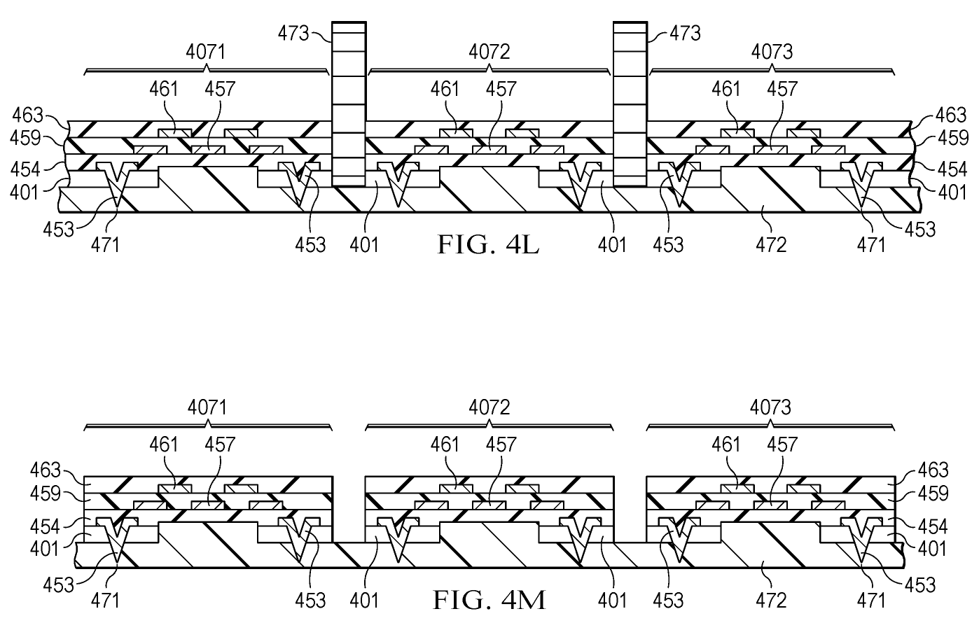

FIGS. 4A-4M illustrate, in a series of cross-sectional views, a method useful for forming a sensor die as the first die for use in an example arrangement. Similar methods for forming the backside metal contacts can be used with other semiconductor die types. The sensor die shown in the illustrated examples is a metal oxide gas sensor. However, the methods and packages of the arrangements can be used with other types of sensor dies, where a sensor material layer is formed and configured to be exposed to the ambient to sense pressure, humidity, strain or stress, water, gas or liquid chemicals or concentrations.

In FIG. 4A, a substrate 401 is shown in a cross-sectional view. The view in FIG. 4A shows a portion of the substrate 401 for a single sensor die, however, in a practical example useful in forming the arrangements, many sensor dies can be formed on substrate 401, which can be a semiconductor wafer. For certain sensor dies, such as metal oxide gas sensors, other substrate materials such as glass substrate can be used. In a particular example, substrate 401 can be a silicon semiconductor wafer.

FIG. 4B illustrates, in a further cross-sectional view, the substrate 401 after a processing step. Trenches 451 are shown formed in an etch process and extending into a device side surface of the substrate 401. In an example process useful for forming an arrangement, a wet silicon etch can be used. Steep trenches 451 can be formed with a V-shape in a cross-section. In alternative shapes useful with the arrangements, diamond, tetrahedron or triangular shaped trench openings can be used, a sharp pointed bottom shape for the trenches 451 is desired to form metal contacts for mounting, in the illustrated arrangements, the metal contacts taper to a point, as is described below.

FIG. 4C illustrates, in a further cross-sectional view, the substrate 401 after an additional step. Conductors 453 are shown deposited in trenches 451 and patterned to form the metal contacts to be used in bonding the sensor die to the second die as described below, or to a wafer including the second semiconductor dies. The conductors 453 can be, for example, copper, aluminum, or gold. In a particular example, aluminum can be used. The conductors 453 will conform to the sides of the trench 451, and in an example may appear as chevron shapes in a cross-section, with tips pointing towards the backside surface of the substrate 401.

FIG. 4D illustrates, in a further cross-sectional view, the substrate 401 after further processing. An insulating layer 454 is shown formed over the substrate 401 and covering the conductors 453. In an example the insulation layer 454 can be an oxide, or a nitride, or a silicon oxynitride, and can be deposited by chemical vapor deposition (CVD) for example.

FIG. 4E illustrates another cross-sectional view that shows the elements of FIG. 4D after an additional processing step. Heater conductors 457 are shown deposited on the layer of insulator 454. In an example useful with the arrangements, a resistive conductor such as tungsten can be used for the heater conductors 457. Tungsten is advantageous for heater conductors as it has a low thermal expansion property (when compared to other conductors used in semiconductor processing) and is stable at relatively high temperatures. During operation, the heater conductors 457 will be supplied with current, and the heater conductors will be used to elevate the sensor die temperature to an operating temperature of between 200-400 degrees C. In sensor die applications where the sensor material does not need to be heated, the heater conductors 457 can be omitted.

FIG. 4F illustrates, in an additional cross-sectional view, the elements of FIG. 4E after a further processing step. In FIG. 4F, the cross-sectional view illustrates the heater conductors 457 after an additional dielectric layer 459 is deposited over the heater conductors. This dielectric layer 459 can be another oxide, nitride, or similar material and can be deposited, for example, by chemical vapor deposition, as in layer 454 was deposited, and can be the same material as layer 454 or layer 459 can be another dielectric.

FIG. 4G illustrates, in a further cross-section, the elements of FIG. 4F, after an additional processing step that forms electrodes 461. In operation, the electrodes 461 will be used to detect changes in conductivity or resistivity of a sensor material (described below) and are spaced from one another. In addition, because the sensor material for a metal oxide layer gas sensor is a thin oxide layer, the electrodes 461 are preferably of a noble conductive material to preclude electromigration or ion migration that can occur with certain conductors, for this reason copper and aluminum are not preferred for the sensor electrodes 461. In an example useful with the arrangements, platinum is used for the sensor electrodes 461. Gold can also be used.

FIG. 4H illustrates, in another cross-sectional view, the elements of FIG. 4G after an additional process deposits a layer of metal oxide material 463 over the sense electrodes 461. This layer of metal oxide material can be deposited, for example, by atomic layer deposition in a reactor chamber, or by chemical vapor deposition. In a particular example, the metal oxide layer 463 is of tin oxide (SnO₂). Other metal oxide materials useful for sensor applications that can form alternative arrangements include titanium oxide (TiO), tungsten oxide (WO₂), indium oxide (In₂O₃), zinc oxide (ZnO), and cobalt oxide (CoO). Various gases can be detected, including some that impact health or cause dangerous conditions for humans, such as CO, CO₂, ethanol, NOx, ammonia, and others. Different metal oxides can be used depending on the target gas, and combinations or bilayers of metal oxides can be used.

Deposition of the metal oxide layer 463 can be by chemical vapor deposition (“CVD”) for example, or by sputtering and oxidation. For example, a tin oxide layer can be used. Tin can be sputtered over the dielectric layer and patterned, and thermal oxidation can be performed.

FIG. 4I illustrates, in another cross-sectional view, the elements of FIG. 4H after a further processing step. In FIG. 4I, the result of a backside etch are shown. The steps described above for FIGS. 4A-4H, including forming the trenches, depositing conductor material in the trenches, forming the insulating layers, the heater conductors, the sensor electrodes and the metal oxide layer, can be performed as “front end of the line “ or “FEOL” processes on the device side surface of a semiconductor substrate (or another substrate) in a wafer fabrication facility.

After the FEOL processes are completed, the wafer can be used in “back end of the line” or “BEOL” processes. In a process used in forming an example arrangement, a silicon etch is performed on the backside surface of the substrate 401 that is selective to the conductor material in the trenches. By removing a portion of the semiconductor substrate from the backside surface, the pointed ends of the conductors in the trenches can be exposed, forming metal contacts 471. Further, in the illustrated example, during the backside etch processes the semiconductor substrate material is removed between the metal contact points 471 to avoid an unwanted current path between the contact points 471 from being formed. The sensor die 407 is now completed.

The metal contacts 471 have pointed or conical ends that taper to a point. Metal contacts 471 are formed extending from the backside surface of the substrate 401 and in examples, the metal contacts 471 have pointed, tapered, V-shaped, cone-shaped, or needle-shaped ends. Use of the metal contacts 471 with pointed ends enables a die-to-die bond between the sensor die and a semiconductor die without the need for mechanical pressure against the sensor die, which can help prevent damage that might otherwise occur to the layer of sensor material, which can be a thin oxide layer or thin layer of material.

The small contact area at the ends of the metal contacts 471 further make alignment to the bond pad or conductive land on the device side surface of the semiconductor die relatively easy to achieve, as there is flexibility in the position of the metal contacts 471 relative to larger bond pads. The sharp pointed ends aid in mounting the sensor dies because by using sonic vibration of the sensor dies during mounting, any thermal or native oxide that is present on the bond pads can be scratched through and a metal-to-metal bond can be made. Various shapes of the metal contacts can be used, including hexagons, octagons, pentagons from a plan view, so long as the distal ends are pointed. In a cross-sectional view, the metal contacts can form an arrowhead, chevron, spear tip or V-shapes such as shown in the illustrated examples. In an example where the metal contacts 471 are not sufficiently low resistance for a given current, additional metal contacts 471 can be provided and coupled in parallel.

FIGS. 4A-4I, described above, illustrate example processes to form a single unit sensor die 407, while FIGS. 4J-4M illustrate, in additional cross-sectional views, a portion of a semiconductor wafer showing some wafer-scale processes that can be used to form sensor dies of the arrangements. In practice, hundreds or even thousands of sensor dies can be formed simultaneously on a wafer or other substrate in a wafer fabrication process.

In FIG. 4J, a semiconductor substrate 401 is shown in a cross-sectional view after the completion of the FEOL processes, with multiple sensor dies 4071, 4072, 4073 formed on the semiconductor substrate 401, which can be a silicon wafer, for example. Each sensor die 4071, 4072, 4073 is identical to the others and includes heater conductors 457, sensor electrodes 461, trench conductors 453, and metal oxide layer 463 over the dielectric layers 459 and 454.

FIG. 4K illustrates the elements of FIG. 4J after additional processing. In FIG. 4K, the semiconductor substrate 401 is shown after a BEOL processing step removes a portion of the semiconductor substrate 401. In an example process that is useful with the arrangements, a plasma silicon etch can be used to remove portions of the semiconductor substrate 401 to expose the metal contacts 471, which were formed by forming conductors in the trenches as described above, and further by removing the semiconductor substrate material 401 between the metal contacts 471 so as to prevent an unwanted current path between the metal contacts from forming in the remaining semiconductor material. The metal contacts 471 are coupled by conductors (not shown for simplicity of illustration) to the heater conductors 457 or to the sensor electrodes 461 to form terminals for the sensor dies. In an example a sensor die (for example any of 4071, 4072, 4073) may have four metal contacts 471, two for supplying the heater conductors with current and two for coupling to the sensor electrodes, or in an alternative arrangement more or less than four terminals can be used.

FIG. 4L illustrates the semiconductor substrate 401 with the sensor dies 4071, 4072, 4073 shown being singulated using scribe streets 4031, 4033 in a wafer dicing operation. The semiconductor substrate 401 is mounted to a dicing tape 472 that supports and stabilizes the semiconductor substrate 401 during processes. A dicing process is shown being performed to cut through the semiconductor substrate 401 along the scribe streets 4031, 4033 between the sensor dies 4071, 4072, 4073. Various dicing processes can be used to dice the semiconductor substrate 401, including mechanical saws 473 as shown in FIG. 4L. Alternative dicing processes include laser dicing, mechanical and laser scribe and break dicing, laser stealth dicing, and plasma dicing.

FIG. 4M illustrates the sensor dies 4071, 4072, 4073 after the singulation process leaves individual units on the dicing tape 472 for processing. The sensor dies 4071, 4072, 4073 correspond to sensor die 407 in FIG. 4I. Each sensor die can be quite small, for example, having an area of 300 microns x 300 microns, even smaller sensor die sizes can be formed using the arrangements. However, it is useful in a production assembly process to make the sensor dies 407 at a size that commonly used automated pick-and-place material handling systems can accommodate, and currently that size is about 300 microns by 300 microns (or more.) Larger sensor dies can also be formed using the arrangements. As additional die transfer technologies become available, smaller sensor dies can be used, increasing the number of units formed from a semiconductor substrate, and reducing the costs of each unit.

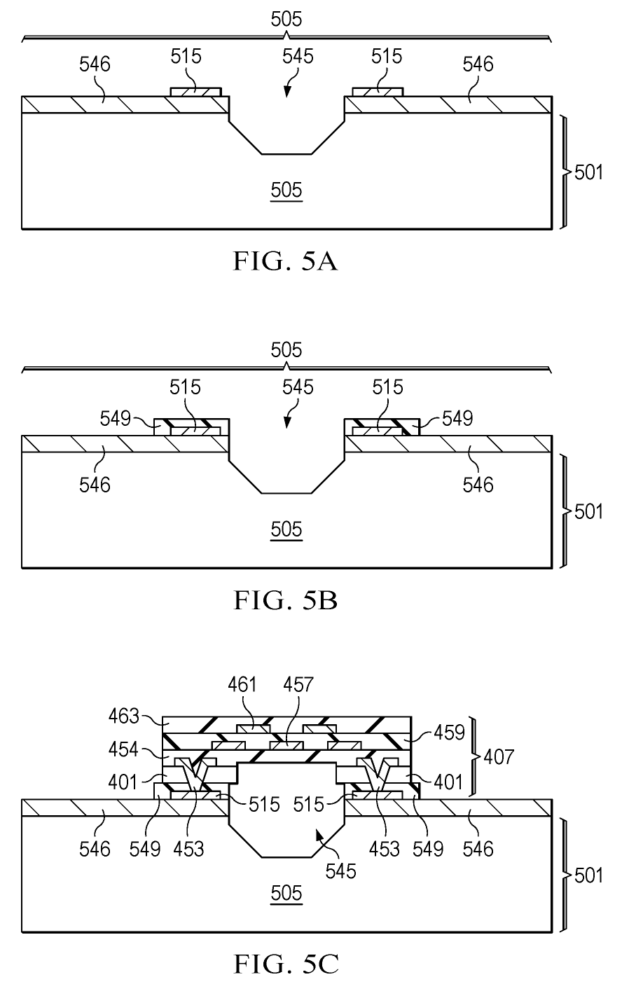

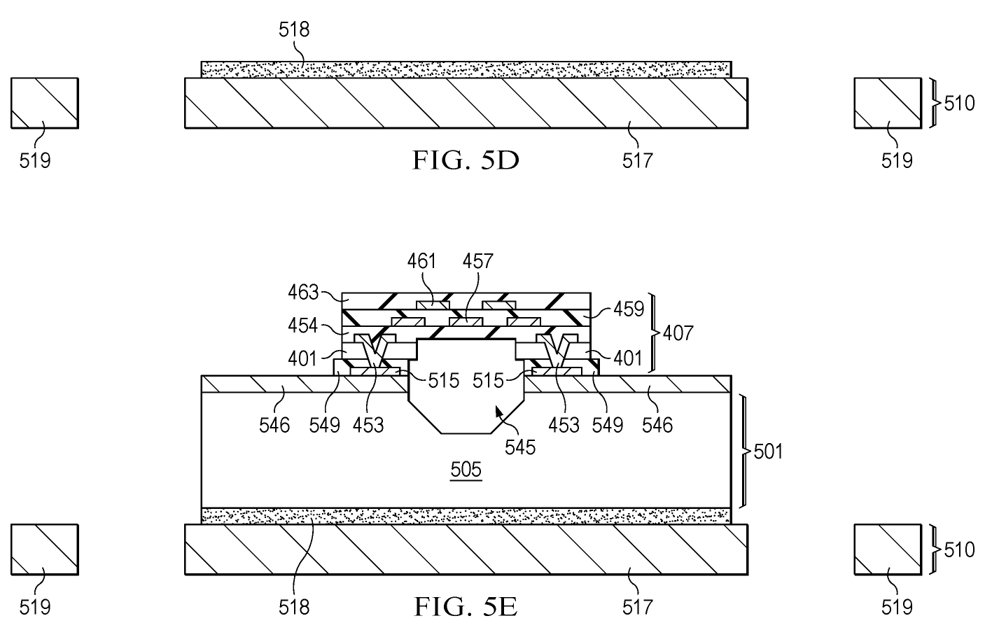

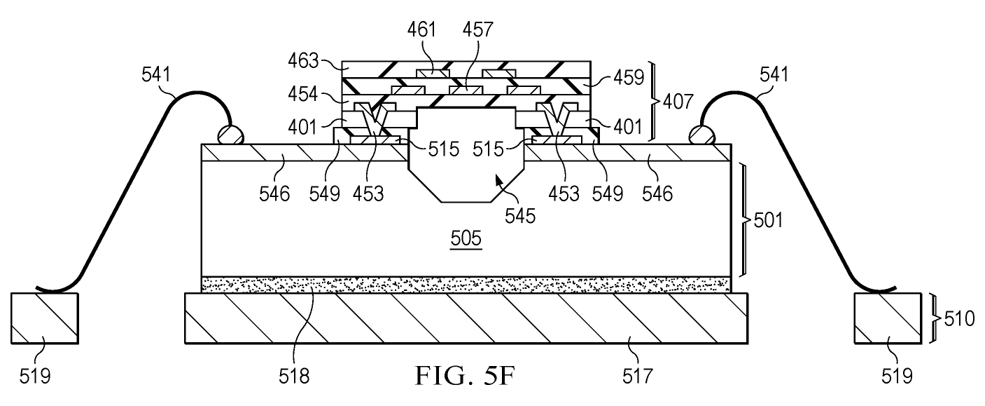

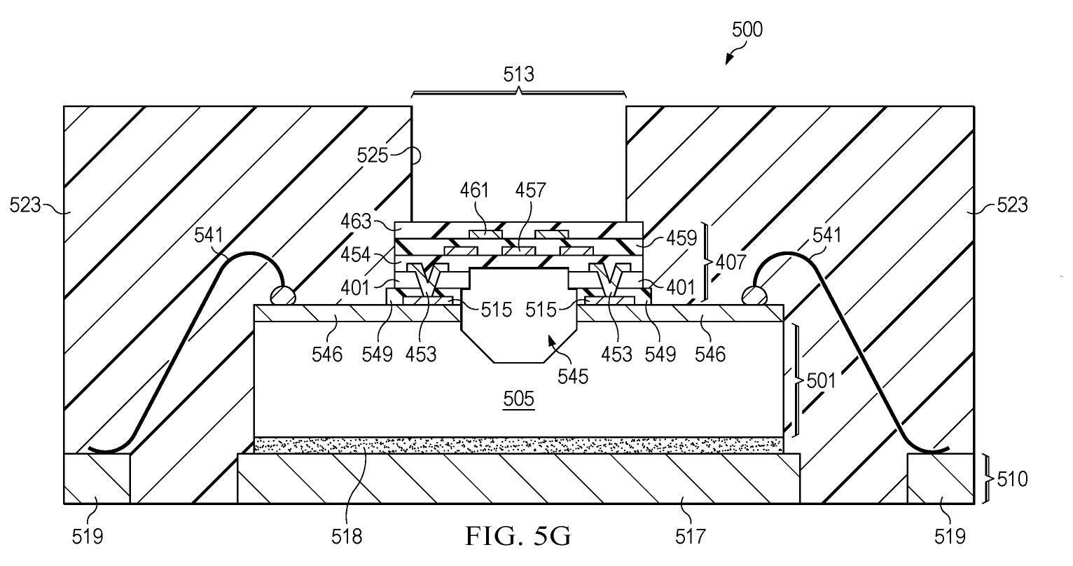

FIGS. 5A-5G illustrate steps that can be used to assemble an example arrangement. In the illustrated steps, the first semiconductor die is shown as a sensor die that is mounted to a second semiconductor die, and the illustrated steps show how to form an example microelectronic device package. FIGS. 5A-5C illustrate, in cross-sectional views, the steps to mount the first die, a sensor die, to the second die, a semiconductor die in the illustrated example, using the backside contacts of the first die. FIGS. 5D-5G illustrate the die mount, wire bonding, and molding steps used to form a microelectronic device package for a device including the combined sensor die and semiconductor die.

However, in additional alternative arrangements, the first semiconductor die and the second semiconductor die can be of die types other than sensors, and certain features of the microelectronic device packages will be different. For example, the sensor dies require an opening exposing a sense material to the atmosphere. Other die types do not require an opening in the mold compound, and so this feature can be omitted, as in FIGS. 1A and 2A. Some first semiconductor dies, including some sensor dies, may not include heater electrodes. In those examples, the heater electrodes can be omitted, and in addition, the thermal isolation trenches shown in the second semiconductor dies can also be omitted. FIGS. 1A-2A, described above, illustrate example microelectronic device packages in arrangements without open cavities, and without the isolation trenches and heater electrodes.

FIG. 5A illustrates in a cross-sectional view a portion of a substrate 501 including a semiconductor die 505, which is at this stage, will still be a portion of substrate 501, which may be a silicon wafer or other semiconductor wafer. A thermal isolation trench 545 is shown formed extending into the device side surface of the semiconductor die 505. In addition, and not shown for clarity of illustration, additional devices such as, for example, transistors, passive components, registers, controller circuitry and the conductors and vias needed to connect these into functional circuits have also been formed in the semiconductor die 505. A protective dielectric layer 546 overlies the device side surface of the semiconductor die 505. Bond pads 515 are shown formed on the semiconductor die 505. Bond pads 515 will be coupled by conductor traces to various components formed with the semiconductor die 505 (not shown for simplicity of illustration). The bond pads 515 are configured to couple to a sensor die, while other bond pads on the semiconductor die 505 (other bond pads are not visible in the portion illustrated) are configured for coupling to leads of a package substrate, for coupling the semiconductor die to package terminals.

FIG. 5B illustrates, in another cross-sectional view, the elements of FIG. 5A after additional processing. In FIG. 5B, the semiconductor die 505 is shown with a patterned adhesive layer 549 deposited over the semiconductor die 505 on the protective dielectric layer 546, with the bond pads 515 having a top side surface (as the elements are oriented in FIG. 5B, the bond pads 515 are facing upwards) exposed from the adhesive 549. The adhesive 549 is arranged around the bond pads 515 so that a sensor die can be later mounted so that the sensor die will adhere to the semiconductor die 505, as is further described below. The adhesive can be a patternable adhesive such as a photo-imageable resin or epoxy composition that is a thermosetting compound. A commercially available photoresist such as SU-8 can be used. After development, the adhesive 549 is left around the bond pads and exposing the top surface of the bond pads 515, ready for mounting the sensor dies to the semiconductor die.

FIG. 5C illustrates, in another cross-sectional view, the elements of FIG. 5B and now including sensor die 407 after a sensor die mounting process mounts the sensor die 407 onto the semiconductor dies, either individually or mounted on substrate 501 as shown. Metal conductors 453 and the contact points (see 471 in FIG. 4I for example) are used to form die-to-wafer bonds or die-to-die bonds between the sensor die 407 and the semiconductor dies such as 505 on the substrate 501. The metal conductors 453 can be of aluminum, copper, or silver or can be silver plated. The bond pads 515 can be of the same material as the metal contacts 471, or, alternatively, a compatible metal where an intermetallic compound will form the bond, such as copper bond pads on the semiconductor die with aluminum metal contacts, or vice versa. If silver (Ag) is used on both the bond pads and the metal contacts, sintering can be used to form the bonds. Further, in another alternative approach, solder plating can be used on the bond pads, and a solder joint can be formed in a thermal reflow process to complete the bonds.

In an example where copper or aluminum bond pads and copper or aluminum metal contacts are used for bond pads 515 and metal contact points 471, ultrasonic or vibration energy can be used to scratch any oxide that may be present on the surface of the bond pads 515 to further improve the bonding process. A pick- and-place tool can be used to place the sensor dies 407 on the corresponding location on the semiconductor dies of the substrate 501.

After the sensor dies such as 407 in FIG. 5C are mounted to the semiconductor dies 505 on the substrate 501, the combined devices can be singulated from the substrate 501, and a semiconductor packaging process can be used to package the combined sensor die and semiconductor die together. FIGS. 5D-5G illustrate, in additional cross-sectional views, steps that can be used to form the packaged device. The semiconductor dies 505 and the sensor dies 407 can be formed independently from one another, at different locations and using different wafer fabrication facilities, and at different times, and the sensor dies can be combined with the semiconductor dies at another facility or at the facility where the semiconductor substrate 501 is processed.

FIG. 5D illustrates, in a cross-sectional view, a package substrate 510, which can be a leadframe such as a copper leadframe, with a die pad 517 and leads 519 spaced from the die pad 517, and a layer of die attach adhesive 518 on a device mounting surface of the die pad 517. The die attach adhesive 518 can be a conductive on non-conductive die attach epoxy, or a die attach film. In an example a non-conductive die attach film is used. The package substrate 510 can include multiple leadframe units provided in a strip, grid, or array, and temporarily connected by tie bars of the package substrate material, which can be removed or cut through later.

FIG. 5E illustrates, in a further cross-sectional view, the elements of FIG. 5D after a die mounting process. The combined semiconductor die 505 and sensor die 407 are singulated from the semiconductor substrate (see 501 in FIG. 5D) and pick and place tools can be used to place the semiconductor die on the die attach material. The die attach material 518 adheres the backside surface of semiconductor die 505 to the die pad 517 of the package substrate 510. While a single unit package substrate 510 is shown for simplicity of illustration, in a practical process for production the package substrate 510 will be one of a strip, array or a grid of units that are provided for contemporaneously mounting semiconductor dies for packaging. Tens, hundreds or more units can be processed contemporaneously to increase throughput and reduce per unit costs for the packaging processes.

FIG. 5F illustrates, in a further cross-sectional view, the elements of FIG. 5E after additional processing. In FIG. 5F, wire bonds 541 are shown forming electrical connections that couple the semiconductor die 505 to the leads 519 of the package substrate 510. In the illustrated example, a no-leads package is being formed, and leads 519 will not extend from the exterior surface of the molded package that is being formed in a subsequent molding process (described below). However, in alternative arrangements, a leaded package can be formed, for example gull-wing shaped leads can be used, and in those alternatives the leads 519 of package substate 510 would include leads that extend farther out from the die pad 517.

In a wire bonding process useful with the arrangements, a ball and stitch wire bonding tool can be used to form the wire bonds 541. In an example process, a wire bonding tool includes a capillary, which can be formed of a hard material such as a ceramic. A bond wire supply such as a spool is provided, and the capillary has an opening in a central portion configured to allow the bond wire to extend through the opening. Clamps associated with or attached to the capillary can be used to selectively hold and to selectively allow the bond wire to extend from the capillary, using the capillary and these clamps the wire bonds can be shaped and positioned as needed. In a wire bonding tool, the capillary can typically move in three directions, in X and Y planes and vertically in a Z plane, to perform the wire bonding process. Alternatively, the workpiece can be moved beneath the capillary in some of those directions, and the capillary can move in the remaining directions.

To form a wire bond, the process begins by forming a molten ball at an exposed end of the bond wire extending from the central opening in the capillary. An electronic arc or flame can be used to form the molten ball. In an example, a copper or copper alloy bond wire can be used. Palladium coated copper (“PCC”) bond wire can be used. Aluminum, silver, or gold bond wire can be used. Copper bond wire is increasingly used for good conductivity at relatively low cost. When copper bond wire is used, the wire bonding tool may include an anoxic environment, for example an inert gas such as nitrogen can be used. An anoxic environment can be used to prevent tarnish and oxidation occurring on the bond pads and on the bond wire, undesirable processes which can be accelerated at the elevated temperatures often used for wire bonding.

The molten ball on the end of the bond wire is then positioned over and placed in contact with a bond pad on a semiconductor die where a wire bond is to be formed. The capillary is used to apply mechanical pressure and ultrasonic vibration to the molten ball to form a mechanical bond to the bond pad. The wire bonder can operate at an elevated temperature to increase the strength of the resulting “ball” bond. The bonding process can be referred to as “thermosonic” reflecting the use of an elevated temperature and sonic energy in conjunction with mechanical force to form the ball bond.

As the capillary moves away from the ball bond, the bond wire is allowed to extend from the capillary and can be shaped in an arc above the edge of the semiconductor die, and the capillary moves to a position over a lead on the package substrate where the wire bond is to end. The capillary again applies mechanical pressure and ultrasonic energy, now to the bond wire, pressing on the lead to form a “stitch” bond using the bond wire. After the stitch bond is formed, the capillary moves a short distance away from the stitch bond and the bond wire is cut or broken. A new free end is exposed on the bond wire extending from the capillary, and the wire bonding tool is ready for the next wire bond cycle. Automated wire bonders can make many wire bonds per second, and all the bonds needed for a given device can be formed in a few seconds, allowing rapid production of the wire bonds for a plurality of semiconductor dies mounted to a package substrate grid or array.

Alternative electrical connections that can be used between the semiconductor die 505 and the package substrate 510 include ribbon bonding and stand-off stitch bond (“SSB”) wire bonds. In SSB bonding, a stand-off bump or ball bond is first formed at a lead or terminal end on a package substrate (or for die to die bonds, another bond pad), and then the ball and stitch bonds are formed starting the bonding cycle as described above, with a ball bond on a bond pad and ending with a stitch bond now made on the stand-off bond.

In an approach for an arrangement formed without wire bonds a flip chip arrangement such as shown in FIGS. 2A-2B, where the semiconductor die is electrically connected to leads using solder balls or conductive post connects, such as copper pillar bumps, instead of wire bonds, can be used to connect to the package substrate. A flip-chip arrangement can be mounted on a package substrate, such as a laminate, a multilayer package substrate, or a build-up multilayer package substrate formed with ABF. Alternatively, a “flip chip on lead” or “FCOL” arrangement can be used where the semiconductor die is flip chip mounted on a leadframe as the package substrate, with conductive post connects or solder balls used to make the electrical connections.

FIG. 5G illustrates, in a further cross-sectional view, the elements of FIG. 5F after additional processing. In FIG. 5G, mold compound 523 is shown forming a body for the microelectronic device package 500 which integrates the semiconductor die 505 and the sensor die 407. Microelectronic device package 500 is similar to the microelectronic device package 100 shown in FIG. 1A, now shown with additional details illustrated. The leads 519 and die pad 517 have board side surfaces that are exposed from the mold compound 523 to form terminals and a thermal pad, respectively, for the microelectronic device package 500. The mold compound 523 has an open cavity 513 positioned in a location that exposes a portion of the sensor material layer 463 on the sensor die 407 to the atmosphere. In the examples this open cavity can be a circular shape (when observed from a top view of the microelectronic device package 500) with sidewalls 525, the open cavity 513 can also have other shapes such as oval, square, or rectangular from a top side view. In a transfer molding operation that can be used to form the mold compound 523, an upper mold chase of a mold for a mold tool can have a portion that contacts the surface of the sensor die 407 and is configured to exclude mold compound 523 from the cavity opening 513 during the transfer molding process. A film assisted mold tool can be used, where a flexible release film is placed in the molds and stretched under a vacuum to conform to the surfaces of the mold chases used in the tool, the release film aids in excluding the mold compound from certain portions and in assists in making the molded package surface smooth and without voids, and aids in releasing the completed devices after molding as well. Electronic mold compound (“EMC”) such as a thermosetting epoxy resin can be used for the mold compound 523.

As described above, the combined semiconductor die and sensor die can be used to form a variety of sensors that use an open cavity package to sense conditions. Gas sensors, acoustic sensors, relative humidity, temperature, liquid, water or rain sensors, strain, and pressure sensors can be formed. Sensor layers that can be used include metal oxide layers, polymer dielectric layers, or piezoelectric layers such as aluminum nitride. BAW and SAW sensors can be formed using sensor materials and conductors on the sensor die.

In additional arrangements, a first die which can be a semiconductor die, a sensor die, or a passive component, can be formed with backside contacts extending from a backside surface, and the first die can be mounted to the device side surface of a second die by forming die-to-die or die-to-wafer bonds between the backside contacts and bond pads on the second die. The second die can be, for example, a semiconductor die and can include circuitry coupled to the first die. Electrical connections are formed between the second die and a package substrate, and mold compound is formed over the first die, the second die, and portions of the package substrate to form a microelectronic device package,

The arrangements provide low cost, reliable and robust assembly by forming the sensor dies with backside metal contacts that are configured for die-to-die or die-to-wafer bonding processes. Use of the arrangements allows vertical stacking of the components without complicated wire bonding or flip chip bonding to bond the sensor dies to the semiconductor dies (as in prior approaches) and provides a small footprint integrated sensor for reduced system board area at reasonable cost.

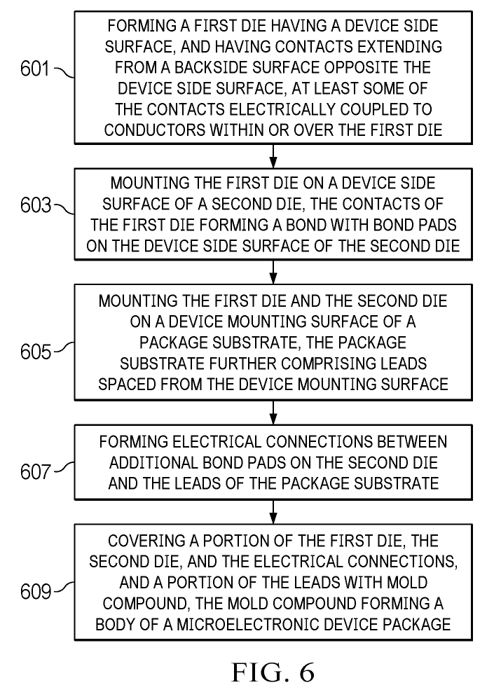

FIG. 6 illustrates, in a flow diagram, steps used to form a microelectronic device package of an arrangement.

At step 601, the method begins by forming a first die having a device side surface and having contacts extending from a backside surface opposite the device side surface, at least some of the contacts electrically coupled to conductors within or over the first die. (See, for example, FIGS. 4A-4I, the first die is 407 in FIG. 4I).

At step 603, the method continues by mounting the first die on a device side surface of a second die, the contacts of the first die forming a bond with bond pads on the device side surface of the second die. (See, for example, FIG. 5C, with the first die 407 mounted on the second die 505).

At step 605, the method continues by mounting the first die and the second die on a device mounting surface of a package substrate, the package substrate further comprising leads spaced from the device mounting surface. (See, for example, FIG. 5E, where the second die 505 is shown mounted on package substrate 510 using die pad 517).

At step 607, the method continues by forming electrical connections between additional bond pads on the second die and the leads of the package substrate. (See, for example, FIG. 5F, where the wire bonds 541 electrically connect the leads 519 to the second die, semiconductor die 505.)

At step 609, the method of FIG. 6 completes by covering a portion of the first die, the second die, and the electrical connections, and a portion of the leads with mold compound, the mold compound forming a body of a microelectronic device package. (See, for example, FIG. 5G, where mold compound 523 is shown forming the body of microelectronic device package 500. See also FIG. 1A, where mold compound 123 forms a microelectronic device package 120, and FIG. 2A, where in a flip chip alternative, mold compound 223 forms the body for a flip chip microelectronic deice package 250.)

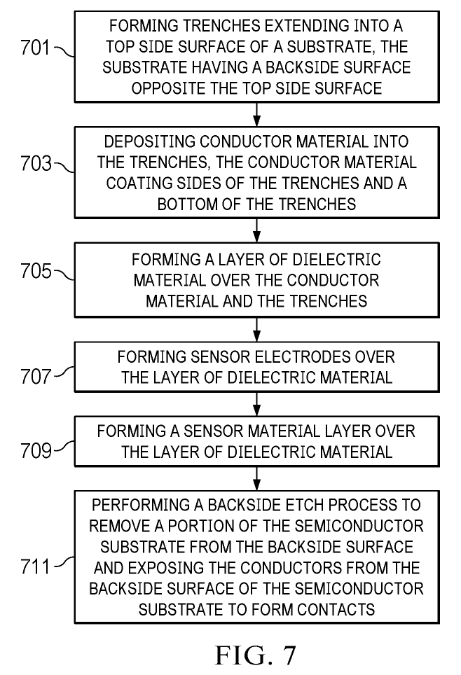

FIG. 7 illustrates, in a flow diagram, the steps used to form a sensor die (see, for example, sensor die 407 in FIG. 4I) as a first die for use in the arrangements.

At step 701, the method begins by forming trenches extending into a top side surface of a substrate, the substrate having a backside surface opposite the top side surface. (See for example, FIGS. 4A-4B with trenches 451 extending into substrate 401 which can be a semiconductor substrate, or another substrate material). The trenches are shaped to have a sharp bottom where sloping sides intersect, the bottom will partially determine the shape of the metal contacts to be formed in subsequent steps.

At step 703, the method continues by depositing conductor material into the trenches, the conductor material coating sides of the trenches and a bottom of the trenches. (See, for example, FIG. 4C where conductor material 453 is shown formed in the trenches). The conductor material can be, for example, copper, a copper alloy, aluminum, or gold. In an example, aluminum was used.

At step 705, the method continues by forming a layer of dielectric material over the conductor material and the trenches. (See, for example, FIG. 4D. layer 454 is shown over the substrate and the trenches and conductors 453).

At step 707, sensor electrodes are formed over the layer of dielectric material. (See, for example, FIG. 4G, where the sensor electrodes 461 are shown formed over the dielectric layers 454, 459). Note that in FIG. 4G, heater conductors 457 are also shown, these are used for gas sensors with thin metal oxide sensor material, but the heater conductors 457 may be omitted for other types of sensors where heating of the sensor material is not necessary.

At step 709, a layer of sensor material is formed over the sensor electrodes. (See, for example, FIG. 4H, where a layer of sensor material 463 is shown formed over the sensor electrodes 461. For example, in a metal oxide gas sensor, the sensor material can be a metal oxide layer. For other sensors, different sensor materials can be used, for example a polymer dielectric can be used for a relative humidity sensor. BAW and SAW sensors can be formed using different sensor materials. Conductor networks formed in or over a piezoelectric can be used for BAW (conductors within the material) and SAW (conductors on the surface of the material) sensors. Conductors over an insulator layer can form a moisture or water sensor, a temperature sensor etc. A photovoltaic cell can be formed using photosensitive material as the sensor material.

At step 711, the method for forming the sensor dies is completed by performing a backside etch process to remove a portion of the substrate from the backside surface and exposing the conductors from the backside to form contacts. (See, for example, FIG. 4I, where contacts 471 are shown exposed from the backside surface of the substrate 401. In an example where a silicon semiconductor substrate is used, the backside etch can be an etch selective to silicon which leaves the conductor material extending from the backside surface.)

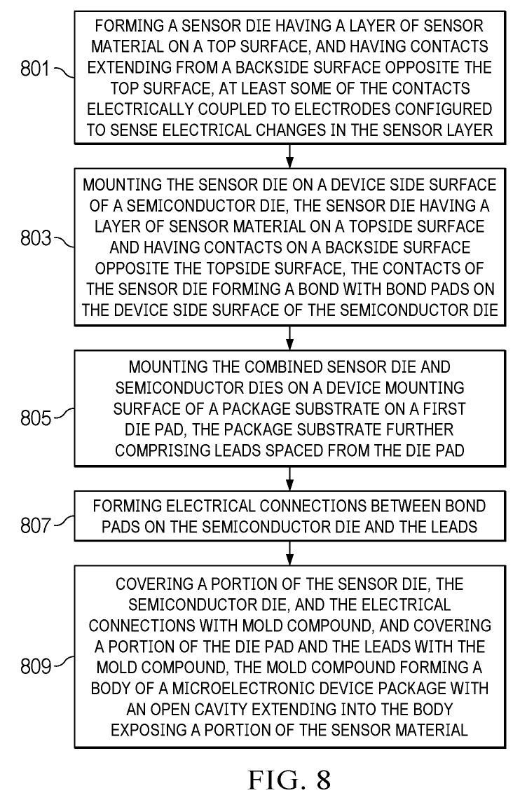

The method of FIG. 7 illustrates, in a flow diagram, steps that can be used to form a sensor die for use as a first die in the arrangements. FIG. 8 illustrates, in a further flow diagram, a method for packaging the sensor die and a semiconductor die together as a first die and a second die vertically arranged in a microelectronic device package. Note that the sensor die (the first die), and the semiconductor die (the second die) can be independently formed at different times, at different facilities, using different semiconductor processes, and at separate locations. There is no order of steps implied by the use of the figure numbers “6”, “7” and “8” in this description.

FIG. 8 illustrates steps to form a microelectronic device package using the sensor die formed in FIG. 7 and a semiconductor die as the second die. The method of FIG. 8 is similar to the method of FIG. 6, but the steps are now arranged for the specific examples where the first die is a sensor die, and the body of the microelectronic device package has an open cavity to expose the sensor die, as shown in FIGS. 1B, 2B and in 5G, for example).

In FIG. 8, the method begins at step 801, by forming a sensor die having a layer of sensor material on a top surface, and having contacts extending from a backside surface opposite the top surface, at least some of the contacts electrically coupled to electrodes configured to sense electrical changes in the sensor layer. (See, for example, FIGS. 4A-4I, where a sensor die 407 is shown being formed in a series of steps, and the method illustrated in FIG. 6, where steps for forming a sensor die are shown in a flow diagram.)

At step 803, the method continues by mounting the sensor die on a device side surface of a semiconductor die, the sensor die having a layer of sensor material on a topside surface and having contacts on a backside surface opposite the topside surface, the contacts of the sensor die forming a die-to-wafer bond with bond pads on the device side surface of the semiconductor die. (See, for example, FIG. 5C, where the sensor die 407 is shown mounted on the device side surface of a semiconductor die 505 which is part of a semiconductor substrate 501.) Sensor dies such as 407 can be mounted to the semiconductor dies while in wafer form in a die-to-wafer bonding process, or, in an alternative approach, the sensor dies 407 can be mounted to the semiconductor dies after a die mounting step, in a die-to-die bonding process.

At step 805, the combined sensor die and semiconductor die are mounted on a device mounting surface of a package substrate on a die pad, the package substrate further comprising leads spaced from the die pad. (See, for example, FIGS. 5D-5E, where a package substrate 510 with a die pad 517 is shown and semiconductor die 505 is shown mounted to the die pad 517 with die attach material 518.)

At step 807, electrical connections are made between bond pads on the semiconductor die and the leads of the package substrate. (See, for example, FIG. 5F, where wire bonds 541 are shown coupling the semiconductor die 505 to the lead 519. See also FIG. 2, where solder balls 147 connect the semiconductor die 105 to the package substrate 212 in a flip chip die mounting arrangement).

At step 809, the method continues by covering a portion of the sensor die, the semiconductor die, and the electrical connections with mold compound, and covering a portion of the die pad and the leads with the mold compound, the mold compound forming a body of a microelectronic device package with an open cavity extending into the body exposing a portion of the sensor material. (See, for example, FIG. 5G, where mold compound 523 is shown forming the body of a microelectronic device package 500, with open cavity 513 exposing a portion of the sensor material 463 of sensor die 407).

The use of the arrangements and methods provide microelectronic device packages including a first die vertically arranged with a second die. In particular examples, the first dies can be sensor dies, and the second dies can be semiconductor dies that are coupled to the sensor dies. The first dies are bonded to the device side surface of the second dies by contacts that extend from the backside surface of the first dies. Use of the arrangements allows for fabricating the first dies, such as the sensor dies, in a wafer manufacturing facility and mounting the sensor dies to the second dies, which can be semiconductor dies, can be done either while the semiconductor dies are part of a semiconductor wafer in a die-to-wafer bonding process, or alternatively after the semiconductor dies are mounted to a package substrate in a die-to-die bonding process. The vertical mounting arrangements of first dies to the second dies provide the ability to package the combined first and second dies in a relatively small package size, especially when compared to the area of multichip module of prior approaches that mount two dies to a laminate or substrate in proximity to one another. The use of the contacts extending from the backside of the first die, such as a sensor die, to mount the first die eliminates the need for additional complex die-to-die wire bonding processes or flip-chip die assembly processes, simplifying the mounting of the first dies to the second dies, and lowering costs.

In some example arrangements, sensor dies are used as the first dies. Various sensor types can be formed using the arrangements where a sensor material is exposed to the environment, including gas sensors, liquid sensors, humidity sensors, temperature sensors, pressure and strain sensors, position sensors, photosensors and acoustic sensors. Sensing layers can include metal oxides, polymer dielectrics, conductor networks over or insulators, photosensitive materials, bulk acoustic wave or surface acoustic wave piezoelectric materials.

Modifications are possible in the described arrangements, and other alternative arrangements are possible within the scope of the claims.

What is claimed is:

1. A method, comprising:
forming a first die having a device side surface, and having contacts extending from a backside surface opposite the device side surface, at least some of the contacts electrically coupled to conductors within or over the first die;
mounting the first die on a device side surface of a second die, the contacts of the first die forming a bond with bond pads on the device side surface of the second die;
mounting the first die and the second die on a device mounting surface of a package substrate, the package substrate further comprising leads spaced from the device mounting surface;
forming electrical connections between additional bond pads on the second die and the leads of the package substrate; and
covering a portion of the first die, the second die, the electrical connections, and a portion of the leads with mold compound, the mold compound forming a body of a microelectronic device package.

2. The method of claim 1, wherein the body of the microelectronic device package further comprises an open cavity extending into the mold compound that forms the body of the microelectronic device package, exposing at least a portion of the first die.

3. The method of claim 2, wherein forming the first die further comprises: forming a sensor die having a layer of sensor material on the device side surface, and having the contacts extending from the backside surface, and at least some of the contacts electrically coupled to sense electrodes configured to sense electrical changes in the layer of sensor material.

4. The method of claim 3, wherein the layer of sensor material on the sensor die further comprises a metal oxide layer, a polymer dielectric layer, a piezoelectric layer configured for a bulk acoustic wave sensor or configured for a surface acoustic wave sensor.

5. The method of claim 3, wherein the layer of sensor material on the sensor die further comprises a metal oxide layer of tin oxide, zinc oxide, titanium oxide or cobalt oxide.

6. The method of claim 3, wherein the layer of sensor material on the sensor die further comprises a polyimide with a dielectric constant that varies with changes in relative humidity.

7. The method of claim 3, wherein the layer of sensor material further comprises aluminum nitride.

8. The method of claim 3, wherein the second die further comprises a semiconductor die, and mounting the first die and the second die on a device mounting surface of a package substrate further comprises forming conductive post connects or solder balls on bond pads of the semiconductor die, and flip chip mounting the semiconductor die onto the device mounting portion of the package substrate.

9. The method of claim 8, wherein forming electrical connections between the additional bond pads on the semiconductor die and the leads further comprises performing a thermal reflow to form solder joints between the conductive post connects or solder balls and the leads of the package substrate.

10. The method of claim 3, wherein the second die is a semiconductor die and wherein mounting the mounting the first die and the second die on a device mounting surface of a package substrate further comprises mounting a backside surface of the semiconductor die to a die pad of the package substrate, the die pad spaced from the leads.

11. The method of claim 10, wherein forming electrical connections between the additional bond pads on the second die and the leads further comprises forming wire bonds.

12. The method of claim 3, wherein forming a sensor die having a layer of sensor material on the device side surface and having the contacts extending from the backside surface further comprises:
forming trenches extending into a top side surface of a substrate, the substrate having the backside surface opposite the top side surface;
depositing conductor material into the trenches, the conductor material coating sides of the trenches and a bottom of the trenches;
forming a layer of dielectric material over the conductor material and the trenches;
forming the electrodes over the layer of dielectric material;
forming the layer of sensor material over the electrodes; and
performing a backside etch process to remove a portion of the substrate from the backside surface and exposing the conductors from the backside surface of the substrate to form the contacts.

13. The method of claim 12, wherein forming the layer of sensor material comprises forming a metal oxide layer, and further comprising:
forming a layer of heater conductors prior to forming the layer of sensor material; and
forming an additional layer of dielectric material over the heater conductors, then subsequently forming the layer of sensor material over the additional layer of dielectric material.

14. The method of claim 12, wherein forming trenches extending into a top side surface of a substrate, the substrate having a backside surface opposite the top side surface further comprises forming the trenches extending into a semiconductor substrate using a wet etch process, wherein the trenches are formed having sloping sides that intersect at a pointed bottom.

15. The method of claim 14, wherein the contacts exposed from the backside surface of the semiconductor substrate have tapered ends.

16. The method of claim 14, wherein the contacts exposed from the backside surface of the semiconductor substrate have pointed, tapered, V-shaped, cone-shaped, or needle-shaped ends.

17. The method of claim 3, wherein the contacts exposed from the backside surface of the first die, the sensor die, comprise copper, aluminum, silver or gold, and the bond pads on the second die are of the same material as the contacts.

18. The method of claim 3, wherein the contacts exposed from the backside surface of the first die, the sensor die, are of aluminum, and the bond pads on the semiconductor die are of copper, and the bond is an intermetallic compound bond.

19. The method of claim 3, wherein the electrodes comprise a noble metal.

20. The method of claim 19, wherein the noble metal is platinum.

21. A method, comprising:
forming a gas sensor die on a substrate, the gas sensor die comprising a metal oxide sensor material over sensor electrodes on a top side surface of the substrate, and further comprising contacts extending from a backside surface of the substrate opposite the top side surface, the sensor electrodes electrically coupled to corresponding ones of the contacts;
forming a semiconductor die on a semiconductor substrate, the semiconductor die having bond pads on a device side surface configured for mounting the gas sensor die;
mounting the semiconductor die on a die pad of a package substrate, the package substrate further comprising leads spaced from the die pad;
mounting the gas sensor die on the device side surface of the semiconductor die, the contacts forming a die-to-die bond with the bond pads on the device side surface of the semiconductor die;
forming wire bond connections between additional bond pads on the semiconductor die and the leads of the package substrate; and
covering a portion of the gas sensor die, the semiconductor die, and the wire bonds with mold compound, and covering a portion of the die pad and the leads with the mold compound, the mold compound forming a body of a microelectronic device package with an open cavity extending into the body exposing the metal oxide sensor material.

22. The method of claim 21, wherein the metal oxide sensor material comprises
tin oxide, zinc oxide, titanium oxide or cobalt oxide.

23. An apparatus, comprising:
a semiconductor die mounted to a device mounting surface of a package substrate, the semiconductor die having bond pads on a device side surface;
a sensor die having a layer of sensor material on a top surface, and having contacts extending from a backside surface opposite the top surface, at least some of the contacts electrically coupled to sensor electrodes configured to sense electrical changes in the layer of sensor material, the sensor die mounted to the device side surface of the semiconductor die using a die-to-die bond between the contacts of the sensor die and the bond pads of the semiconductor die;
electrical connections formed between additional bond pads on the device side surface of the semiconductor die and leads of the package substrate; and
mold compound covering a portion of the sensor die, the semiconductor die, and the electrical connections, the mold compound also covering a portion of the leads and forming a body of a microelectronic device package with an open cavity extending into the body exposing the layer of sensor material.

24. The apparatus of claim 23, wherein the layer of sensor material further comprises a metal oxide layer, a polymer dielectric layer, a photosensitive material layer, or a piezoelectric material layer.

25. The apparatus of claim 24, wherein the layer of sensor material further comprises a metal oxide layer of tin oxide, zinc oxide, titanium oxide or cobalt oxide.

26. A microelectronic device package, comprising:
a first die having devices formed within or over a device side surface of a substrate and further comprising contacts extending from a backside surface of the substrate opposite the device side surface;
a second die mounted over a die pad of a package substrate, the package substrate further comprising leads spaced from the die pad, the second die comprising a semiconductor die having bond pads on a device side surface configured for mounting the first die, and having additional bond pads;
the first die mounted to the second die by die-to-die bonds formed between the contacts and the bond pads configured for mounting the first die;
electrical connections between additional bond pads on the device side surface of the second die and leads of the package substrate; and
mold compound covering at least a portion of the first die, the second die, the electrical connections, a portion of the die pad and a portion of the leads, the mold compound forming a body of a microelectronic device package.

27. The microelectronic device package of claim 26, wherein the first die comprises a sensor die, and further comprising an open cavity extending into the body of the microelectronic device package exposing a portion of a sensor material on the device side surface of the sensor die.

28. The microelectronic device package claim 27, wherein the sensor die further comprises a gas sensor die and the sensor material further comprises a metal oxide sensor material of tin oxide, zinc oxide, titanium oxide or cobalt oxide.

29. The microelectronic device package of claim 28, wherein the sensor die further comprises heater conductors formed in insulator material between the substrate of the gas sensor die and the metal oxide sensor material.

30. The microelectronic device package of claim 26, wherein the first die is a sensor die comprising a layer of sensor material that is a metal oxide layer, a polyimide with a dielectric constant that varies with changes in relative humidity, or a layer of aluminum nitride, and further comprising an open cavity extending into the body of the microelectronic device package exposing a portion of the layer of sensor material on the sensor die.
